(19) United States
(12) Patent Application Publication
(10) Pub. No.: US 2024/0304355 A1
(43) Pub. Date: Sep. 12, 2024

(54) METAL NANOWIRE FOAM

(71) Applicant: MONASH UNIVERSITY, CLAYTON, Victoria (AU)

(72) Inventors: Wenlong CHENG, Clayton, Victoria (AU); Lim Wei YAP, Clayton, Victoria (AU); Fenge LIN, Clayton, Victoria (AU); Kaixuan WANG, Clayton, Victoria (AU)

(21) Appl. No.: 18/275,827

(22) PCT Filed: Feb. 4, 2022
(86) PCT No.: PCT/AU2022/050060

§ 371 Date: Aug. 4, 2023

(30) Foreign Application Priority Data
Feb. 5, 2021 (AU) 2021900277

(51) Int. Cl. H01B 1/22; A61B 5/00; A61B 5/257; A61B 5/28; C23C 18/16; C23C 18/20; C23C 18/44; C25B 3/07; C25B 3/09; C25B 11/031; C25B 11/052; C25B 11/057; C25B 11/081; H01Q 1/36

(52) U.S. Cl. CPC H01B 1/22; A61B 5/0006; A61B 5/257; A61B 5/28; C23C 18/1641; C23C 18/1644; C23C 18/1666; C23C 18/2086; C23C 18/44; C25B 11/031; C25B 11/052; C25B 11/057; C25B 11/081; H01Q 1/364; A61B 2560/0214; C25B 3/07; C25B 3/09

(57) ABSTRACT

Deformable porous elastic conductors and fabrication methods thereof, as well as their use in a broad range of applications including electrodes, supercapacitors, antennae, and electrocatalysts, medical devices, soft electronic devices and wearable sensors.

### TECHNICAL FIELD

The present invention relates to the field of deformable porous elastic conductors and fabrication thereof.

In one form, the invention relates to deformable porous elastic conductors suitable for use as sensors.

In some forms, the invention relates to deformable porous elastic conductors for use in a broad range of applications ranging from electrodes, supercapacitors, antennae, and electrocatalysts to medical devices, soft electronic devices and wearable sensors.

It should be appreciated that the present invention is not limited to only to the specific uses described herein, and that it can be applied to a wide range of medical (including veterinary) and non-medical uses, including industrial uses.

### BACKGROUND ART

The following discussion of the background art is intended to facilitate an understanding of the present invention only. The discussion is not an acknowledgement or admission that any of the material referred to is or was part of the common general knowledge as at the priority date of the application.

Soft electronics require a seamless combination of deformability and conductivity, requiring the design of soft/hard materials interfaces, which tend to fail leading to delamination and/or cracks due to mechanical mismatching. The Young's moduli of conductively active materials including metals, semiconductors, carbons, and conducting polymers, are typically a few orders of magnitude higher than those of elastomeric polymers and other deformable substrates. Interfacing the brittle conductively active materials with flexible substrates or supports poses significant challenges in providing durable soft electronics that are easily manufactured at industrial scales. Many of the currently known designs of such systems are limited in their durability and suffer the drawbacks of delamination and/or cracks at the soft/hard materials interface when subjected to high and/or repetitive strains.

Substantial efforts have been made by recent researchers to deposit or embed optoelectronic materials into/onto elastomeric materials in pursuit of soft electronic components.^{1,2} In this context, 2D elastomeric sheets and 1D elastomeric fibers have been widely used as substrates for integrating with active materials such as carbon, metal, silicon and conducting polymers for developing various soft electronic systems.³⁻⁷ In addition, elastomeric sponges, such as polyurethane sponge (PU sponge), have also been exploited in soft electronics,⁸⁻¹⁰ in which 3D porous frameworks, light-weightness, and mechanical elasticity can be combined with electrical conductivity leading to novel sensors and energy devices.¹⁻¹³

The dip-coating approach represents the dominant strategy to fabricate conductive sponges due to its simplicity. With this approach, conductive inks, such as those comprising CNT (Carbon Nanotubes),^{6,11,13} carbon black,¹⁵ graphene,^{16,17} or silver nanowires^{9,18} have been successfully used for constructing conductive sponges. In addition, freeze-drying represents another strategy to fabricate conductive sponges. For example, copper nanowire aerogels^{11,24} have been successfully obtained by mixing PVA [Poly(vinyl alcohol)] and copper nanowire solutions followed freeze-drying. Silver nanowire,²⁰ graphene,²¹ and CNT-rGO (Carbon Nanotube—Reduced Graphene Oxide)²² aerogel conductive sponges were achieved by directly freeze-drying these suspensions. In addition, polymer-assisted copper deposition¹⁰ and gold ion sputter-based metallic sponges¹² have also been reported in the literature.

Despite this encouraging progress, some challenging issues remain. The dip-coating approach typically suffers from poor adhesion between the conductive fillers and the sponge skeleton support; carbon-based sponges typically only offer low conductivity; ion-sputtered metallic films experience cracking with resultant reduction of conductivity under mechanical deformation.

Low-cost processes for manufacturing durable, deformable elastic conductors comprising porous 3-D elastomeric substrates, amenable to large-scale production, would enable the utilisation of such conductors in a broad range of applications ranging from electrodes, supercapacitors, antennae, and electrocatalysts to medical devices, soft electronic devices and wearable sensors.

Consequently, there is a need to develop high performance deformable porous elastic conductors that circumvent, overcome or obviate one or more of the abovementioned limitations.

Further challenges and particular difficulties are posed in implementing the use of soft electronic components to a broad range of applications ranging from electrodes, supercapacitors, antennae, and electrocatalysts to medical devices, soft electronic devices and wearable sensors.

Consequently, there is a need to develop high performance deformable elastic conductors for use in a broad range of sensing applications, that circumvent, overcome or obviate one or more of the abovementioned limitations.

It is against this background that the present invention has been developed.

### SUMMARY OF THE INVENTION

Provided herein is a simple, scalable and efficient electroless metal nanowire coating technology enabling the fabrication of highly electrically-conductive, deformable, porous, elastic, compressible, mechanically soft, and catalytically active materials with broad applications as strain-insensitive conductors, soft supercapacitors, catalysts, sensors including wireless sensors, battery-free sensors and powered sensors, soft dry electrodes, antennae, and in a multitude of sensing applications including in implementations of medical devices and wearable biodiagnostic devices.

In one embodiment, the invention described herein provides a deformable porous elastic conductor comprising;
a 3D porous elastomeric substrate, wherein a plurality of the surfaces of the 3D porous elastomeric substrate are covalently functionalised with complexing moieties; and
a plurality of metal nanowires, each complexed to at least one of the complexing moieties, wherein the metal nanowires are upstanding, relative to the surface to which they are attached via their respective complexing moiety.

In some embodiments, the metal nanowires comprise a nanoparticle head and a nanowire tail.

In some embodiments, the metal nanowires comprise a metal selected from the group consisting of gold, platinum, palladium, rhodium, copper, silver, ruthenium, osmium, iridium, rhenium, iron, cobalt, nickel, zinc, manganese, titanium, vanadium, chromium, molybdenum, tungsten, magnesium, lead and aluminium.

In some embodiments, the metal nanowires comprise a noble metal.

In a preferred embodiment, the metal nanowires comprise gold.

In some embodiments, the deformable porous elastic conductor is compressible.

In some embodiments, the deformable porous elastic conductor is biocompatible.

In some embodiments, the deformable porous elastic conductor is chemically inert.

In some embodiments, the 3D porous elastomeric substrate of the deformable porous elastic conductor is a sponge, or a synthetic polymer sponge, or a polyurethane sponge.

In some embodiments, the complexing moieties of the deformable porous elastic conductor are amine groups.

In some embodiments, the plurality of the surfaces of the 3D porous elastomeric substrate are covalently functionalised with an (Aminoalkyl)trialkyloxysilane, or (3-Aminopropyl)trimethoxysilane or (3-Aminopropyl)triethoxysilane.

In some embodiments, the deformable porous elastic conductor of the invention has a conductivity which is insensitive to compression, bending or twisting.

In some embodiments, the deformable porous elastic conductor of the invention has a linear region of response to strain when measured as relative change in resistance (ΔR/Rₒ) with strain or relative change in current (ΔI/Iₒ) with strain.

In some embodiments, the deformable porous elastic conductor of the invention has a linear region of response to strain when measured as relative change in resistance (ΔR/Rₒ) with strain or relative change in current (ΔI/Iₒ) with strain that is particularly advantageous in applications as a strain sensor.

In some embodiments, the deformable porous elastic conductors of the invention have a linear region of response to strain when measured as relative change in resistance (ΔR/Rₒ) with strain or relative change in current (ΔI/Iₒ) with strain that is tunable by varying the concentration of nanowire growth solution when fabricating the deformable porous elastic conductors.

In some embodiments, the deformable porous elastic conductor of the invention has an insensitivity to compressive strain as measured by increase in resistance, of 3% or less at up to 80% compressive strain.

In some embodiments, the deformable porous elastic conductor of the invention has an insensitivity to bending as measured by increase in resistance, of 4% or less at up to 1800 bending.

In some embodiments, the deformable porous elastic conductor of the invention has an insensitivity to twisting as measured by increase in resistance, of 0.6% or less at up to 3600 twisting.

In some embodiments, the deformable porous elastic conductor of the invention has a conductivity of 1500 S m⁻¹ or better, at 5 min metal nanowire growth time, or a conductivity of 5500 S m⁻¹ or better, at 15 min metal nanowire growth time.

In some embodiments, the deformable porous elastic conductor of the invention has either; A. a conductivity of 1500 S m⁻¹ or better, at 5 min metal nanowire growth time, or a conductivity of 5500 S m⁻¹ or better, at 15 min metal nanowire growth time; and/or insensitivity to tensile strain as measured by relative resistance (R/Rₒ) of 15% or less at up to 44% strain; and/or insensitivity to compressive strain as measured by relative change in resistance (ΔR/Rₒ), of 42% or less at up to 80% compressive strain; and/or insensitivity to bending as measured by relative change in resistance (ΔR/Rₒ), of 8% or less at up to 180° bending; and/or insensitivity to twisting as measured by relative change in resistance (ΔR/Rₒ), of 21% or less at up to 10800 twisting; and/or insensitivity to washing with aqueous detergent solution as measured by relative change in resistance (ΔR/Rₒ), of 26% or less at up to 10 cycles of washing with aqueous detergent solution; and/or insensitivity to tape stripping tests as measured by relative change in resistance (ΔR/Rₒ), of 14% or less at up to 10 cycles of tape stripping test; and/or insensitivity to scratch tests as measured by relative change in resistance (ΔR/Rₒ), of 41% or less at up to 10 cycles of scratch test; and/or insensitivity to rubbing tests as measured by relative change in resistance (ΔR/Rₒ), of 50% or less at up to 10 cycles of rubbing test; or B. a linear region of response to tensile strain when measured as relative change in resistance with strain (ΔR/Rₒ), in the range of 30-50% tensile strain, or 50-70% tensile strain, or 10-70% tensile strain; and/or a linear region of response to compressive strain when measured as relative change in current (ΔI/Iₒ) with compressive strain, in the range of 5 kPa to 38 kPa; preferably with a sensitivity within the linear region of 8.42 kPa-1.

In some embodiments, the deformable porous elastic conductor of the invention is embedded in a soft elastomeric material, PDMS elastomer, or addition cure silicone rubber.

In some embodiments, the deformable porous elastic conductor of the invention embedded in a soft elastomeric material, PDMS elastomer, or addition cure silicone rubber exhibits improved stretchability and/or is stretchable up to approximately 340% without loss of conductivity and/or without significant deterioration in conductivity.

In some embodiments, the deformable porous elastic conductor embedded in a soft elastomeric material, PDMS elastomer, or addition cure silicone rubber has an insensitivity to tensile strain as measured by relative resistance (R/Rₒ) of 1.26 or less at up to 60% tensile strain and 1.83 or less at up to 100% tensile strain; or has an insensitivity to tensile strain as measured by relative resistance (R/Rₒ) of 1.3 or less at up to 60% tensile strain and 1.9 or less at up to 100% tensile strain.

In some embodiments, the deformable porous elastic conductor of the invention has a conductivity retention of >95% after 5000 stretch-release cycles at 30% strain; or is highly durable, as determined by 12% or less changes in conductivities under 5000 stretch-release cycles at 30% strain.

In some embodiments, the deformable porous elastic conductor of the invention is used as a soft electronic device.

In some embodiments, the deformable porous elastic conductor of the invention is used as a sensor, or a wearable sensor.

In some embodiments, the deformable porous elastic conductor of the invention is used as a soft inductive-capacitive sensor.

In one embodiment, the deformable porous elastic conductor of the invention is used as an electrocatalyst for catalysing chemical reactions.

In some embodiments, there is a provided an electrode, a supercapacitor, an antenna, or an electrocatalyst comprising the deformable porous elastic conductor of the present invention.

In some embodiments, the porous elastic conductor of the present invention is used as a dry soft electrode.

In one embodiment, the porous elastic conductor of the present invention is used as a dry soft electrode for skin interfacing applications.

In some embodiments, there is a provided a data collection device, or an Electrocardiograph (ECG) device, or an Electromyograph (EMG) device, or an Electroencephalograph (EEG) device, comprising the deformable porous elastic conductor of the present invention.

In some embodiments, there is a provided a wearable data collection device, or an Electrocardiograph (ECG) device, or an Electromyograph (EMG) device, or an Electroencephalograph (EEG) device, comprising the deformable porous elastic conductor of the present invention.

In some embodiments, there is a provided a wearable data collection device, or an Electrocardiograph (ECG) device, or an Electromyograph (EMG) device, or an Electroencephalograph (EEG) device, comprising the deformable porous elastic conductor of the present invention, wherein the device is capable of wirelessly transmitting data to a separate data logging and processing device.

In one embodiment, the device is a wearable Electrocardiograph (ECG) device, capable of wirelessly transmitting data to a separate data logging and processing device.

In some embodiments, the device comprises a dry soft electrode comprising the deformable porous elastic conductor of the invention, wherein the electrode maintains a stable electrical resistance of 10 for over 30 days of use.

In some embodiments, the device comprises a dry soft electrode comprising the deformable porous elastic conductor of the invention, wherein the electrode has a thickness of approximately 2 mm, or a thickness of less than approximately 2 mm, or a thickness of approximately 1.5 mm, or a thickness of less than approximately 1.5 mm, or a thickness of approximately 1 mm, or a thickness of less than approximately 1 mm.

In some embodiments, the device comprising the deformable porous elastic conductor of the present invention;
a) comprises an ultrathin battery, having a thickness of not more than 1 mm; and/or
b) comprises a flexible circuit board, comprising at least one microprocessor and a wireless transmitter; and/or
c) comprises a soft flexible adhesive for attaching the device to a user, or a subject, or a surface from which data is to be collected; and/or
d) is not more than 6.1 cm long, not more than 2.6 cm wide and not more than 4 mm thick; and/or
e) is reusable, cleanable and sanitisable.

In one embodiment, the present invention provides a method of fabricating the deformable porous elastic conductor of the invention, the method comprising the steps of;
(i) optionally, pre-treating the 3D porous elastomeric substrate;
(ii) functionalising the 3D porous elastomeric substrate with a functionalising agent;
(iii) seeding the functionalised 3D porous elastomeric substrate with metal nanoparticles; and
(iv) growing metal nanowires from the metal nanoparticles.

In some embodiments, of the method of the invention;
(i) pre-treating the 3D porous elastomeric substrate in accordance with step (i) comprises air plasma treatment; and/or
(ii) functionalising the 3D porous elastomeric substrate in accordance with step (ii) comprises introducing a functionalising agent in the presence of the application of sonication and/or the application of negative pressure to facilitate infiltration or penetration of the functionalising agent into the 3D porous elastomeric substrate; and/or
(iii) seeding the functionalised 3D porous elastomeric substrate with metal nanoparticles in accordance with step (iii) comprises introducing a seed solution comprising metal nanoparticles and optionally a stabiliser, optionally in the presence of the application of sonication and/or the application of negative pressure to facilitate infiltration or penetration of the seed solution into the 3D porous elastomeric substrate; and/or
(iv) growing metal nanowires from the metal nanoparticles in accordance with step (iv) comprises introducing a growth solution comprising a metal salt, a reducing agent and a surfactant or ligand, optionally in the presence of the application of sonication and/or the application of negative pressure to facilitate infiltration or penetration of the growth solution into the 3D porous elastomeric substrate.

In some embodiments, of the method of the invention the functionalising agent is an (Aminoalkyl)trialkyloxysilane, or (3-Aminopropyl)trimethoxysilane, or (3-Aminopropyl)triethoxysilane.

In some embodiments, the functionalisation agent is an alcoholic solution of (Aminoalkyl)trialkyloxysilane, or (3-Aminopropyl)trimethoxysilane or (3-Aminopropyl)triethoxysilane.

In some embodiments, the functionalisation agent is a solution of (Aminoalkyl)trialkyloxysilane, or (3-Aminopropyl)trimethoxysilane or (3-Aminopropyl)triethoxysilane, in methanol, or in ethanol, or in isopropanol.

In some embodiments, the functionalisation agent is an aqueous solution of (Aminoalkyl)trialkyloxysilane, or (3-Aminopropyl)trimethoxysilane or (3-Aminopropyl)triethoxysilane.

In some embodiments, the functionalisation agent is a solution of (Aminoalkyl)trialkyloxysilane, or (3-Aminopropyl)trimethoxysilane or (3-Aminopropyl)triethoxysilane, in water.

In some embodiments, the functionalisation agent is a solution of (Aminoalkyl)trialkyloxysilane, or (3-Aminopropyl)trimethoxysilane or (3-Aminopropyl)triethoxysilane, in a mixture of alcoholic solvent and aqueous solvent.

In some embodiments, the deformable porous elastic conductor covalently functionalised with an aqueous solution of functionalising agent is more insensitive to compression, bending or twisting than the deformable porous elastic conductor covalently functionalised with an alcoholic solution of functionalising agent.

In some embodiments, of the method of the invention;
(i) seeding the functionalised 3D porous elastomeric substrate with metal nanoparticles in accordance with step (iii), comprises seeding the functionalised 3D porous elastomeric substrate with gold nanoparticles; and
(ii) growing metal nanowires from the metal nanoparticles in accordance with step (iv), comprises growing gold nanowires, via exposure of the 3D porous elastomeric substrate to a solution comprising HAuCl₄, L-ascorbic acid and 4-mercaptobenzoic acid.

In some embodiments, of the method of the invention, the growth of the nanowires is tuned by fabricating a series of the deformable porous elastic conductors with varying concentrations of growth solution.

In comparison to prior art approaches involving dip-coating, freeze-drying and ion-sputtering, the fabrication strategy of the present invention simultaneously offers the advantages of conformal coating, robust metal-elastomer bonding interfaces and electroless deposition under ambient conditions, achieving strain-insensitive conductivity, durable maintenance of outstanding capacitance even under extreme mechanical compression and bending, robust and recyclable catalysis, a wide range of linear responses to applied pressure in battery-free pressure sensing applications, as well as significant advantages in terms of durability, shelf-life, stable and low impedance, re-usability, and minimisation of signal noise due to motion artefacts when implemented in wearable biodiagnostic devices compared to existing gel-based electrodes typically used in such applications.

The soft deformable porous elastic conductors of the invention also demonstrate applicability to a myriad of technical applications in future intelligent systems in which soft sensors and energy devices are required.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is; (A) A Schematic diagram of the process for the synthesis of metal nanowires on a 3D porous elastomeric substrate; (B-D) SEM images of a metal nanowire (v-AuNWs) deformable porous elastic conductor in accordance with the present invention, wherein the scale bar is 500 μm, 5 μm and 500 nm respectively.

FIG. 2 is; (A) A plot of the relative change in resistance of the metal nanowire (v-AuNWs) deformable porous elastic conductor under different tensile strains; (B) A plot of the relative change in resistance of the metal nanowire (v-AuNWs) deformable porous elastic conductor under different compression strains; (C) A plot of the relative change in resistance of the metal nanowire (v-AuNWs) deformable porous elastic conductor under different degrees of twisting strain. (D) A plot of the relative change in resistance of the metal nanowire (v-AuNWs) deformable porous elastic conductor under different degrees of bending strain.

FIG. 3 is; (A) A plot of the relative change in resistance of the metal nanowire (v-AuNWs) deformable porous elastic conductor under 10 cycles of washing with detergent; (B) A plot of the relative change in resistance of the metal nanowire (v-AuNWs) deformable porous elastic conductor under 10 cycles tape test (C) A plot of the relative change in resistance of the metal nanowire (v-AuNWs) deformable porous elastic conductor under 10 cycles of scratching with fingernail; (D) A plot of the relative change in resistance of the metal nanowire (v-AuNWs) deformable porous elastic conductor under 10 cycles of rubbing with finger.

FIG. 4 is; (A) I-V curves of the metal nanowire (v-AuNWs) deformable porous elastic conductor before and after Ecoflex is embedded; (B) Stretchability plot showing relative change in resistance with tensile strain of the Ecoflex embedded metal nanowire (v-AuNWs) deformable porous elastic conductor (the inset is the magnification of relative resistance change of the conductor up to 200% tensile strain); (C) I-V curves of the Ecoflex embedded metal nanowire (v-AuNWs) deformable porous elastic conductor at different stretching strain levels; (D) Durability plot of the Ecoflex embedded metal nanowire (v-AuNWs) deformable porous elastic conductor at 30% stretching strain showing stable resistance after 5000 cycles of stretching.

FIG. 5 is; (A) Resistance plot of the Ecoflex embedded metal nanowire (v-AuNWs) deformable porous elastic conductor under different stretch strains increasing from 10% to 100%; (B) I-V curves of the Ecoflex embedded metal nanowire (v-AuNWs) deformable porous elastic conductor at different stretching strains up to 200%.

FIG. 6 is; SEM images of the metal nanowire (v-AuNWs) deformable porous elastic conductor before (A) and after (B) pseudocapacitive polyaniline (PANI) deposition, scale bar is 500 nm.

FIG. 7 is; (A) Schematic illustration of a supercapacitor comprising the metal nanowire (v-AuNWs) deformable porous elastic conductor in accordance with the present invention; (B) CV curves of the supercapacitor at various scan rates; (C) GCD curves of the supercapacitor under various currents; (D) Volume specific capacitances of the supercapacitor at different scan rates; (E) CV curves at 200 mV s⁻¹ of the supercapacitor under different compression strains from 0 to 50%; (F) CV curves at 200 mV s⁻¹ of the supercapacitor under different degrees of bending strain from 0 to 180°.

FIG. 8 is; A plot of the % retention of capacitance of a supercapacitor comprising the metal nanowire (v-AuNWs) deformable porous elastic conductor in accordance with the present invention showing >80% retention with up to 2000 cycles of CV tests at 200 mV s⁻¹.

FIG. 9 is; (A) GCD curves of the supercapacitor under different compression strains; (B) Capacitance retention of the supercapacitor under different compression strains; (C) Capacitance retention under repeated compression of the supercapacitor at 50% for 1000 cycles; (D) CV curves of the supercapacitor under 200 mV s⁻¹ before and after 1000 compression cycles of 50% compression.

FIG. 10 is; (A) GCD curves of the supercapacitor under different degrees bending of bending strain; (B) Capacitance retention of the supercapacitor under different degrees of bending strain; (C) Capacitance retention of the supercapacitor under repeated bending to 180° for 1000 cycles; (D) CV curves of the supercapacitor under 200 mV s⁻¹ before and after 1000 bending cycles of bending to 180°.

FIG. 11 is; (A) Chemical equation for the reduction of p-nitrophenol by NaBH₄ by a metal nanowire (v-AuNWs) deformable porous elastic electrocatalyst; (B) The absorbance spectra during reduction of p-nitrophenol in aqueous solution by the electrocatalyst, recorded at several intervals; (C) Plot of the % conversion efficiency after 10 cycles showing the reusability of the electrocatalyst for the reduction of 4-nitrophenol by NaBH₄; (D) The relationship between ln(Cₜ/C₀) and reaction time (t) for the first and tenth reaction cycles of the electrocatalyst.

FIG. 12 is; SEM images of the metal nanowire (v-AuNWs) deformable porous elastic electrocatalyst in accordance with the present invention before (A) and after (B) catalysis, scale bar is 500 nm.

FIG. 13 is; A schematic of the fabrication process of an antenna comprising the metal nanowire (v-AuNWs) deformable porous elastic conductor in accordance with the present invention;

FIG. 14 is; (a) Schematic of the experimental setup for the measurement of the performance of an antenna comprising Ecoflex encapsulated metal nanowire (v-AuNWs) deformable porous elastic conductor in accordance with the present invention; (b) Plot of phase change of reader coil with varying applied pressures; (c) Plot of changes in resonant frequency of reader coil with increasing applied pressure.

FIG. 15 is; A plot of the measured maximum phase dip of reader coil versus the detection distance between the coil and the antenna comprising Ecoflex encapsulated metal nanowire (v-AuNWs) deformable porous elastic conductor having an elastic conductor ribbon thickness of 8.5 mm; 0.01° is background noise level and the signal amplitude that is higher than this can be regarded as an effective signal.

FIG. 16 is; A comparison of practical and computational results (a-c) plottingresonant frequency versus applied pressure in different situations; (a) Experimental result; (b) Simulated result; (c) Theoretical result. The pressure values at the lowest resonant frequencies are indicated with arrows; (d) Schematic illustration of the sponge antenna's structure with geometrical parameters.

FIG. 17 is; A diagram showing the antenna comprising elastomer encapsulated metal nanowire (v-AuNWs) deformable porous elastic conductor in accordance with the present invention under (a) Initial relaxed state and (b) Under compression state.

FIG. 18 is; A diagram depicting a SONNET simulation; (a-b) 2D/3D view of antenna model for the antenna comprising elastomer encapsulated metal nanowire (v-AuNWs) deformable porous elastic conductor in accordance with the present invention; (c) Dielectric layers and (d) metal types setup for the antenna under a pressure of 122 kPa.

FIG. 19 is; A series of plots of; (a) Theoretical inductance and capacitance versus pressure for the sponge antenna encapsulated in Ecoflex. The inductance and capacitance were calculated based on equations 4 and 5 described herein; (b) Theoretical ε_{r-eff} versus pressure; (c) Theoretical K(ξ₀′/K(ξ₀) versus pressure; (d) Theoretical elastic conductor ribbon length versus pressure. This length is calculated based on Poisson effect after calculating the compression ratio of sponge antenna's thickness.

FIG. 20 is; a series of plots showing the effect of elastomer rigidity of the encapsulation polymer and ribbon thickness of the metal nanowire (v-AuNWs) deformable porous elastic conductor on the position of transition point; (a-c) Measured resonant frequency as a function of applied pressure for sensors with different encapsulation elastomer rigidity where the antenna was encapsulated in PDMS; For the PDMS, the mixing ratio (w/w) of base and curing agent is (a) 20:1, (b) 30:1 and (c) 40:1; The thickness of the metal nanowire (v-AuNWs) deformable porous elastic conductor ribbon is 8.5 mm; (d) Measured resonant frequency as a function of applied pressure for sensors with different ribbon thickness of the metal nanowire (v-AuNWs) deformable porous elastic conductor thickness; The ribbon thickness is 3 mm, 6.5 mm and 8.5 mm; For the PDMS, the mixing ratio of base and curing agent is 30:1.

FIG. 21 is; A plot depicting the durability test for the antenna encapsulated in PDMS; The mixing ratio of base and curing agent is 30:1 and the sponge thickness is 8.5 mm; 100 kPa and 300 kPa pressure were applied to the antenna for 25 cycles, respectively; During each cycle, static pressure was continuously applied to the antenna for 27 s and then removed for 27 s; In each 27 s period, 17 s is for adequate response time for the network analyser and 10 s is the time taken to obtain a stable resonant frequency signal; (b) Magnified view of (a).

FIG. 22 is; A scheme for demonstrating the application of measuring human body weight in accordance with the present invention; For the experimental setup, three sponge antennae with same area and rigidity were placed under a glass board (5 kg) and the reader coil was placed under the leftmost antenna. The sponge antennae were encapsulated in PDMS with a sponge ribbon thickness of 8.5 mm and the mixing ratio of PDMS is 30:1. During the test, three volunteers with body weights of 61.3, 66.7 and 80.7 kg, stood on the glass board, respectively.

FIG. 23 is; A demonstration of the utility of the antenna comprising Ecoflex encapsulated metal nanowire (v-AuNWs) deformable porous elastic conductor in accordance with the present invention, in measuring body weight; (a) Comparison of the antenna responses to people with different body weight in a static loading situation; (b) The resonant frequency changes of the antenna when people with different weights stood on it one by one to test the function of measuring people's weight in dynamic loading situation.

FIG. 24 is; A schematic depicting attachment of a wearable ECG device on the chest of a user or subject and the wireless transmission of data being collected by a wearable ECG device comprising a dry metal nanowire (v-AuNWs) deformable porous elastic electrode in accordance with the present invention, to a separate data logging and processing device (eg; a mobile phone), and an expanded schematic view of a wearable ECG device comprising a dry metal nanowire (v-AuNWs) deformable porous elastic electrode in accordance with the present invention; the device has a flexible single lead ECG module paired with 1 mm ultrathin battery, with an overall size of 6.1 cm×2.6 cm and thickness of 4 mm, including the dry electrode and soft flexible sealing adhesive bandage layer.

FIG. 25 is; A plot of electrical resistance change of metal nanowire (v-AuNWs) deformable porous elastic conductors grown on sponge functionalised with (3 Aminopropyl)triethoxysilane dissolved in (a) ethanol and (b) water, under a tensile strain range of 2.5%, 5%, 10%, 15%, 20% to 25%; (c) The gauge factor of both samples as a function of tensile strain (2.5%-25%); The durability performance of the metal nanowire (v-AuNWs) deformable porous elastic conductor grown on sponge functionalised with (3 Aminopropyl)triethoxysilane diluted with (d) ethanol and (e) water, under 10% tensile strain for 1000 cycles.

FIG. 26 is; A of plot electrical resistance change of metal nanowire (v-AuNWs) deformable porous elastic conductors grown on sponge functionalised with (3 Aminopropyl)triethoxysilane dissolved in (a) ethanol and (b) water under various compression strains ranging from 1 kPa, 2 kPa, 3 kPa, 5 kPa, 7.5 kPa to 10 kPa; (c) The sensitivity of both samples as a function of pressure (1 kPa-10 kPa); The durability performance of the metal nanowire (v-AuNWs) deformable porous elastic conductor grown on sponge functionalised with (3 Aminopropyl)triethoxysilane dissolved in (d) ethanol and (e) water under 5 kPa compressive strain for 1000 cycles.

FIG. 27 is; (A) A plot comparing the stretchability of the v-AuNWs grown on 2D ecoflex elastomeric substrate functionalized with (3-Aminopropyl)trimethoxysilane diluted with water and ethanol, as a function of increase in resistance with applied tensile strain; (B) A photographic image showing a large visible crack under 200% strain for v-AuNWs grown on 2D ecoflex elastomeric substrate functionalized with an ethanol solution of APTMS; (C) A photographic image showing no visible cracks or damage under 800% strain for v-AuNWs grown on 2D ecoflex elastomeric substrate functionalized with an aqueous solution of APTMS.

FIG. 28 is; A schematic depicting attachment of the round dry metallic nanowire (v-AuNWs) deformable porous elastic electrode cut into diameter of 12 mm and thickness of 1.5 mm onto the flexible ECG circuit board using a conductive adhesive.

FIG. 29 is; A diagram depicting attachment of a metal nanowire (v-AuNWs) deformable porous elastic electrode in accordance with the present invention vs a rigid metallic dry electrode on a non-uniform curvilinear surface such as the chest of a user or subject, when used in an ECG or other type of electrophysiological monitoring device such as an electromyogram (EMG), electroencephalogram (EEG), electrodermal activity (EDA) for example.

FIG. 30 is; A series of plots comparing the ECG signal collected using a dry metal nanowire (v-AuNWs) deformable porous elastic electrode in accordance with the present invention (a,c,e), and a commercial Ag/AgCl gel electrode (b,d,f), during (a,b) sleep, (c,d) computer work and (e,f) walking.

FIG. 31 is; A plot comparing the skin-electrode impedance of the dry metal nanowire (v-AuNWs) deformable porous elastic electrode and a commercial gel-electrode.

FIG. 32 is a series of plots depicting; (A) Impedance change (top) and phase change (bottom) of gold nanowire foam after applying tensile/stretching (40%), compression (80%), and twisting (1080°) strain in artificial sweat solution; and (B) Comparison of the electrode impedance (top) at 1 kHz and relative resistance changes (bottom) of commercial gel electrode and gold nanowire foam electrode before and after aging in artificial sweat solution for up to 1 week. The impedance is normalized to the impedance value before aging.

FIG. 33 is a series of plots depicting tensile strain sensing performance of v-AuNWs PU sponge fabricated with; (A) 25 vol %; (B) 50 vol %; (C) 75 vol %; and (D) 100 vol % of AuNWs growth solution.

FIG. 34 is a series of plots depicting compressive strain sensing performance of v-AuNWs PU sponge fabricated with; (A) 25 vol %; (B) 50 vol %; (C) 75 vol %; and (D) 100 vol % of AuNWs growth solution.

### DEFINITIONS

Throughout this specification, unless the context requires otherwise, the word “comprise” or variations such as “comprises” or “comprising”, will be understood to imply the inclusion of a stated integer or group of integers but not the exclusion of any other integer or group of integers.

As used herein, the term “and/or” includes any and all combinations of one or more of the associated listed items.

As used herein, the term “deformable” means a material that is flexible such that it allows its shape to be temporarily changed when a force is exerted upon it, and that will substantially revert to its original shape once the force is no longer exerted. For example, deformable materials may include, but are not limited to, silicone, EPDM rubber, nylon, synthetic polymers, elastomers, polyurethane, as well as equivalents and combinations thereof.

As used herein, the term “insensitive”, and grammatical variations thereof such as “insensitivity” (etc), shall be understood to refer to the property of the deformable elastic conductors of the present invention whereby they continue to function as electrical conductors at substantially the same level of performance when they are deformed, including when they are deformed under compressive strain, or tensile strain, or twisting strain, or bending strain etc.

As used herein, the term “deformation-insensitive conductivity” (and grammatical variations of the phrase) refers to the property of a deformable conductor in accordance with the present invention having a minimal, or negligible or functionally insignificant (in the context of the functions required by the applications in which the present invention finds use) change in electrical resistance/conductivity/impedance when being deformed, including being deformed under compressive strain, or tensile strain, or twisting strain, or bending strain etc.

It should be understood that “deformation-insensitive conductivity” does not mean that the deformable elastic conductors of the present invention do not necessarily exhibit any sensitivity to strain or deformation. The person skilled in the art will understand that a preferably linear response to changes in relative resistance (ΔR/Rₒ) with strain and/or a preferably linear response to changes in relative current (ΔI/Iₒ) with strain, is a necessary and favourable property in certain embodiments of the deformable elastic conductors of the present invention, that lends them to applications as sensors, including but not limited to tensile strain sensors and compressive strain (pressure) sensors. Accordingly, as used herein, the term “sensitivity”, when used in the context of the present invention, will be understood to refer to sensitivity in the application of the deformable elastic conductors of the present invention as sensors. Certain embodiments of the deformable elastic conductors of the present invention possess a linear region of response to strain when measured as relative change in resistance (ΔR/Rₒ) with strain or relative change in current (ΔI/Iₒ) with strain. For example, certain embodiments of the present invention possess a linear region of response to tensile strain when measured as relative change in resistance with strain (ΔR/Rₒ), in the range of 30-50% tensile strain, or 50-70% tensile strain, or 10-70% tensile strain, or a linear region of response to compressive strain when measured as relative change in current (ΔI/Iₒ) with compressive strain, in the range of 5 kPa to 38 kPa; certain embodiments possess a sensitivity within the linear region of response to compressive strain of 8.42 kPa⁻¹.

As used herein, the term “strain insensitive deformable elastic conductor” refers to the deformable elastic conductors of the present invention having the property of deformation-insensitive conductivity. For example, certain embodiments of the present invention possess an insensitivity to tensile strain as measured by relative resistance (R/Rₒ) of 1.26 or less at up to 60% tensile strain and 1.83 or less at up to 100% tensile strain, or an insensitivity to compressive strain as measured by increase in resistance, of 3% or less at up to 80% compressive strain, or an insensitivity to bending as measured by increase in resistance, of 4% or less at up to 1800 bending, or an insensitivity to twisting as measured by increase in resistance, of 0.6% or less at up to 360° twisting. Certain embodiments of the present invention possess an insensitivity to tensile strain as measured by relative resistance (R/Rₒ) of 15% or less at up to 44% strain, or an insensitivity to compressive strain as measured by relative change in resistance (ΔR/Rₒ), of 42% or less at up to 80% compressive strain, or an insensitivity to bending as measured by relative change in resistance (ΔR/Rₒ), of 8% or less at up to 1800 bending, or an insensitivity to twisting as measured by relative change in resistance (ΔR/Rₒ), of 21% or less at up to 10800 twisting, or an insensitivity to washing with aqueous detergent solution as measured by relative change in resistance (ΔR/Rₒ), of 26% or less at up to 10 cycles of washing with aqueous detergent solution, or an insensitivity to tape stripping tests as measured by relative change in resistance (ΔR/Rₒ), of 14% or less at up to 10 cycles of tape stripping test, or an insensitivity to scratch tests as measured by relative change in resistance (ΔR/Rₒ), of 41% or less at up to 10 cycles of scratch test, or an insensitivity to rubbing tests as measured by relative change in resistance (ΔR/Rₒ), of 50% or less at up to 10 cycles of rubbing test.

As used herein, the term “porous” when referring to a substrate, product or material means a substrate, product or material that has accessible and interconnected voids located therein such that there exist pathways through which a fluid may pass, extending through the entire thickness of the material.

As used herein, the term “wearable” broadly refers to devices associated with the user or subject, e.g. worn over or attached to a body part, or surface, or embedded into an item of clothing or footwear, and configured for sensing of various parameters of the user or subject. In this context, the subject may be human or non-human.

As used herein, the term “wirelessly” refers to a communication path from a source to a destination (e.g., between two devices). Wireless communication may occur via any number of means that are well known in the art, including, but not limited to, Bluetooth™, WiFi™, cellular network or other means of radio transmission.

As used herein, the “vertically aligned metal nanowires” grown on 3D porous elastomeric substrates in accordance with the invention may be referred to generally in abbreviated form as “v-MNWs”. For more specific examples, where the vertically aligned metal nanowires are platinum nanowires, they may be referred to as “v-PtNWs”, where the vertically aligned metal nanowires are gold nanowires, they may be referred to as “v-AuNWs”, where the vertically aligned metal nanowires are palladium nanowires, they may be referred to as “v-PdNWs”, etc.

As used herein, the terms “encapsulated” and “embedded” as they pertain to the encapsulation or embedment of the deformable porous elastic conductor of the present invention in a solid elastomeric material, are used interchangeably and should be understood to have the same meaning in this context.

As used herein, the term “conductive adhesive” refers to any type of adhesive that is electrically conductive, for example, without limitation, silver paste, graphite paste, copper tape, carbon tape, et cetera.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### DETAILED DESCRIPTION

Electronics is evolving from rigid, flexible to ultimate stretchable electronics in which active optoelectronic materials are required to deposit onto or embedded into elastomeric materials. The present invention herein demonstrates a powerful solution-based electroless metal coating technology, which enables growth of enokitake-like metal nanowires on three-dimensional (3D) porous elastomeric substrates for a wide of applications in soft electronics, medical devices, wearable bioelectronics, soft electrodes, soft supercapacitors, sensing antenna, and electrocatalysis.

Herein is disclosed a direct conformal electroless metal-coating strategy to grow highly conductive metal nanowire films uniformly throughout porous elastomeric 3D substrates via facile yet powerful metal nanowire growth protocols.

Unlike conventional metal-coating technologies fabricated by evaporation or electrodeposition, the metal films of the present invention exhibit enokitake-like Janus morphologies, leading to unconventional optical, wetting, electrical, electrochemical and mechatronic properties depending on the surfaces of the substrate that are exposed to the process.

The present disclosure demonstrates that metal nanowire growth technology can be extended to 3D porous elastomeric substrates, achieving uniform conformal coating of metal nanowires with a conductivity of 1500 S m⁻¹, or more, with up to 5500 S m⁻¹ after a metal nanowire growth time of 15 minutes.

The deformable porous elastic conductors of certain embodiments of the invention are insensitive to external deformations including compression, bending, and twisting, showing only a 3% increase in resistance at 80% compression strain, 4% increase at 1800 bending degree and 0.6% increase at 360° twisting degree.

The deformable porous elastic conductors of certain embodiments of the invention are insensitive to external deformations including insensitivity to tensile strain as measured by relative resistance (R/Rₒ) of 15% or less at up to 44% strain; and/or insensitivity to compressive strain as measured by relative change in resistance (ΔR/Rₒ), of 42% or less at up to 80% compressive strain; and/or insensitivity to bending as measured by relative change in resistance (ΔR/Rₒ), of 8% or less at up to 1800 bending; and/or insensitivity to twisting as measured by relative change in resistance (ΔR/Rₒ), of 21% or less at up to 10800 twisting; and/or insensitivity to washing with aqueous detergent solution as measured by relative change in resistance (ΔR/Rₒ), of 26% or less at up to 10 cycles of washing with aqueous detergent solution; and/or insensitivity to tape stripping tests as measured by relative change in resistance (ΔR/Rₒ), of 14% or less at up to 10 cycles of tape stripping test; and/or insensitivity to scratch tests as measured by relative change in resistance (ΔR/Rₒ), of 41% or less at up to 10 cycles of scratch test; and/or insensitivity to rubbing tests as measured by relative change in resistance (ΔR/Rₒ), of 50% or less at up to 10 cycles of rubbing test.

The deformable porous elastic conductors of certain embodiments of the invention possess a linear region of response to strain when measured as relative change in resistance (ΔR/Rₒ) with strain or relative change in current (ΔI/Iₒ) with strain. In preferred embodiments the deformable porous elastic conductors of the present invention possess a linear region of response to tensile strain when measured as relative change in resistance with strain (ΔR/Rₒ), in the range of 30-50% tensile strain, or 50-70% tensile strain, or 10-70% tensile strain; and/or a linear region of response to compressive strain when measured as relative change in current (ΔI/Iₒ) with compressive strain, in the range of 5 kPa to 38 kPa; preferably with a sensitivity within the linear region of 8.42 kPa-1.

Further embedding deformable porous elastic conductors in a solid elastomeric material, or an addition cure silicone rubber, such as but not limited to EcoFlex, in accordance with the invention improves the stretchability of the porous elastic conductors up to ˜100%.

The deformable porous elastic conductors embedded in Ecoflex silicone rubber have high-performance strain-insensitive properties that only increase up to 17.3% in resistance under 50% tensile strain, and up to 83.3% in resistance under 100% tensile strain, while being able to be stretched up to ˜340% under tensile strain before losing conductivity.

This strain-insensitive property in conjunction with the high conductivity and porous structure of the present invention, motivated the present inventors to design and implement outstanding supercapacitors with a capacitance of up to 127.3 mF cm⁻³ showing almost no performance deterioration even under up to 50% compressive strain and up to angles of 1800 bending strain.

Interestingly, the present inventors have found that the deformable porous elastic conductors of the present invention could efficiently catalyse 4-nitrophenol into 4-aminophenol under ambient conditions with 90% efficiency even after 10 reaction cycles.

Thus, the present invention provides a multifunctional conductive soft materials platform for a multitude of future sensing, catalysis and energy applications.

### Deformable Porous Elastic Conductors on 3D Porous Elastomeric Substrates

In one embodiment, the present invention provides a deformable porous elastic conductor comprising; a 3D porous elastomeric substrate, wherein a plurality of the surfaces of the 3D porous elastomeric substrate are covalently functionalised with complexing moieties; and a plurality of metal nanowires, each complexed to at least one of the complexing moieties, wherein the metal nanowires are upstanding, relative to the surface to which they are attached via their respective complexing moiety. In some embodiments the metal nanowires comprise a nanoparticle head and a nanowire tail.

In some embodiments, the metal nanowires comprise a metal selected from the group consisting of gold, platinum, palladium, rhodium, copper, silver, ruthenium, osmium, iridium, rhenium, iron, cobalt, nickel, zinc, manganese, titanium, vanadium, chromium, molybdenum, tungsten, magnesium, lead and aluminium.

In some embodiments, the metal nanowires comprise a noble metal, preferably gold.

In some embodiments the deformable porous elastic conductor is compressible, and/or biocompatible, and/or chemically inert, and/or possesses the property of having a conductivity of which is insensitive to compression, bending or twisting.

In one embodiment, the disclosure herein provides a method of fabricating the deformable porous elastic conductor of the present invention, the method comprising the steps of; optionally, pre-treating the 3D porous elastomeric substrate; functionalising the 3D porous elastomeric substrate with a functionalising agent; seeding the functionalised 3D porous elastomeric substrate with metal nanoparticles; and growing metal nanowires from the metal nanoparticles.

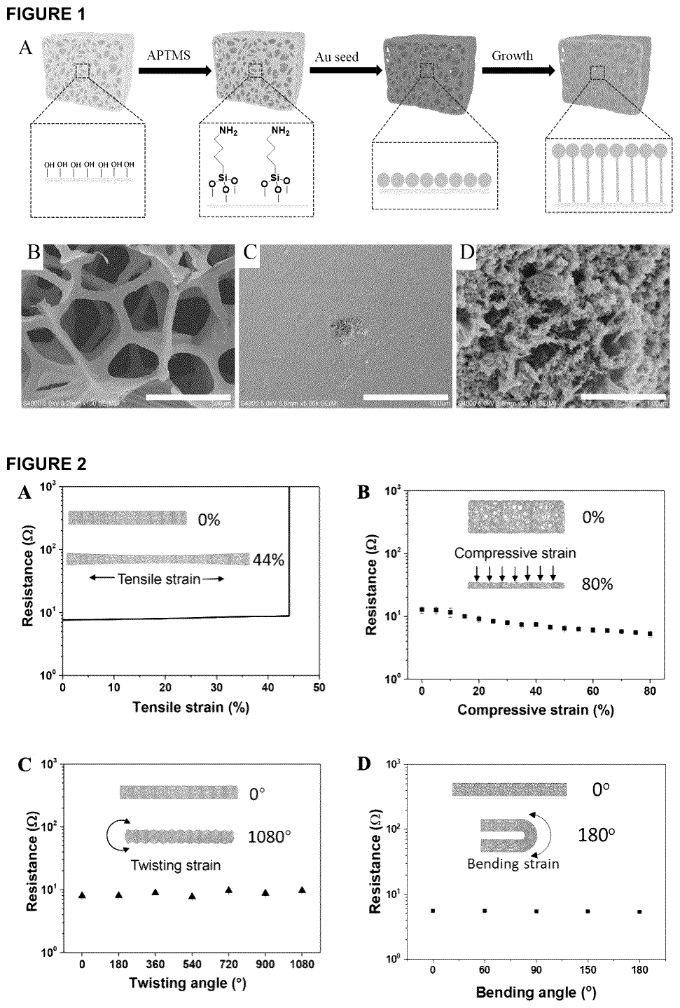

FIG. 1A illustrates the deformable porous elastic conductor preparation process. Firstly, 3D porous elastomeric substrate (eg; polyurethane sponge) is pre-treated via air plasma to render its surfaces hydrophilic, followed by surface functionalisation with a functionalising agent. In a representative embodiment, the surface of the substrate was functionalised via amine modification using (3-aminopropyl)trimethoxysilane (APTMS) as functionalising agent in a hydrolysis reaction.

The skilled addressee will understand that any functionalising agent capable of forming a covalent bond with the hydroxyl groups of the 3D elastomeric substrate via an analogous hydrolysis reaction, and possessing a complexing moiety capable of complexation to metal nanoparticles may be employed in accordance with the present invention. Alternative functionalisation agents include, but are not limited to, for example, any (Aminoalkyl)trialkyloxysilane, such as (3-Aminopropyl)triethoxysilane, or (3-Aminopropyl)tripropoxysilane.

Step 2 of FIG. 1A illustrates the complexation of metal nanoparticles to the complexing moieties of the functionalised 3D elastomeric substrate. In a representative embodiment, the amine-functionalized polyurethane sponge was immersed into a seed solution containing gold nanoparticles for 2 h. This step led to the attachment of gold seeds onto the PU sponge substrate based on electrostatic attraction leading to complexation to the amine groups of the amine-functionalized polyurethane sponge.

The skilled addressee will understand that a seed solution comprising any metal nanoparticles capable of complexing to the complexing moieties of the functionalised 3D porous elastomeric substrate may be used in accordance with the present invention. Alternative seed solutions include, but are not limited to, for example, seed solutions comprising suspensions of metal nanoparticles of platinum, palladium, rhodium, copper, silver, ruthenium, osmium, iridium, rhenium, iron, cobalt, nickel, zinc, manganese, titanium, vanadium, chromium, molybdenum, tungsten, magnesium, lead or aluminium.

Step 3 of FIG. 1A illustrates the growth of metal nanowires on the metal nanoparticle seeded functionalised 3D elastomeric substrate. In a representative embodiment, immersion of gold seed-complexed polyurethane (PU) sponge into a growth solution containing metal nanowire precursors of an appropriate metal salt (Gold (III) chloride trihydrate), ligand (4-Mercaptobenzoic acid) and reducing agent (L-ascorbic acid), induced growth of enokitake-like vertically aligned gold nanowires (v-AuNWs) perpendicular to the surfaces of the polyurethane sponge substrate.

The skilled addressee will understand that a growth solution comprising any metal nanowire precursors may be used in accordance with the present invention. For example, alternative metal salts, and/or alternative ligands, and/or alternative reducing agents may be employed without departing from the general principle of the invention.

The fabrication process is entirely solution-based. Unlike physical deposition of metallic film techniques such as sputter coating, the main advantage of this solution-based method is that v-MNWs can conformally grow throughout porous 3D sponge substrate. The scanning electron microscope (SEM) images of as-synthesized v-AuNWs sponge are presented in FIGS. 1B-D under different magnifications. FIG. 1B is the skeleton of v-AuNWs sponge, and shows that the v-AuNWs coating is uniform and did not affect the porous structure of the PU sponge. With higher resolution as shown in FIG. 1C and FIG. 1D, one can clearly identify enokitake-like nanowire structures on the skeleton of PU sponge substrate. The estimated length of the v-AuNWs is about 120 nm.

The gold coating changed the optical appearance from light yellow to brown. Nevertheless, the deposition of v-AuNWs did not alter mechanical properties of the PU sponge. The gold sponge retained excellent elastic properties and could withstand folding and compressing. The further stress-strain characterization of PU sponge and v-AuNWs sponge demonstrated similar mechanical properties between the two. The v-AuNWs sponge showed 15% or less change in the electrical resistance while being stretched up to 44% of strain, and lost its conductivity completely when stretched beyond 44% (FIG. 2A).

The conductivity of the deformable porous elastic conductors of the invention possesses remarkable insensitivity to deformation under compression (FIG. 2B), twisting (FIG. 2C), and bending (FIG. 2D) as measured in terms of change in resistance with deformation. Typically, the resistance of the v-AuNWs sponge has 42% or less relative change in resistance under 80% compressive strain, 21% or less relative change in resistance under 10800 degrees of twisting strain and 8% or less relative change in resistance under 1800 degrees of bending strain.

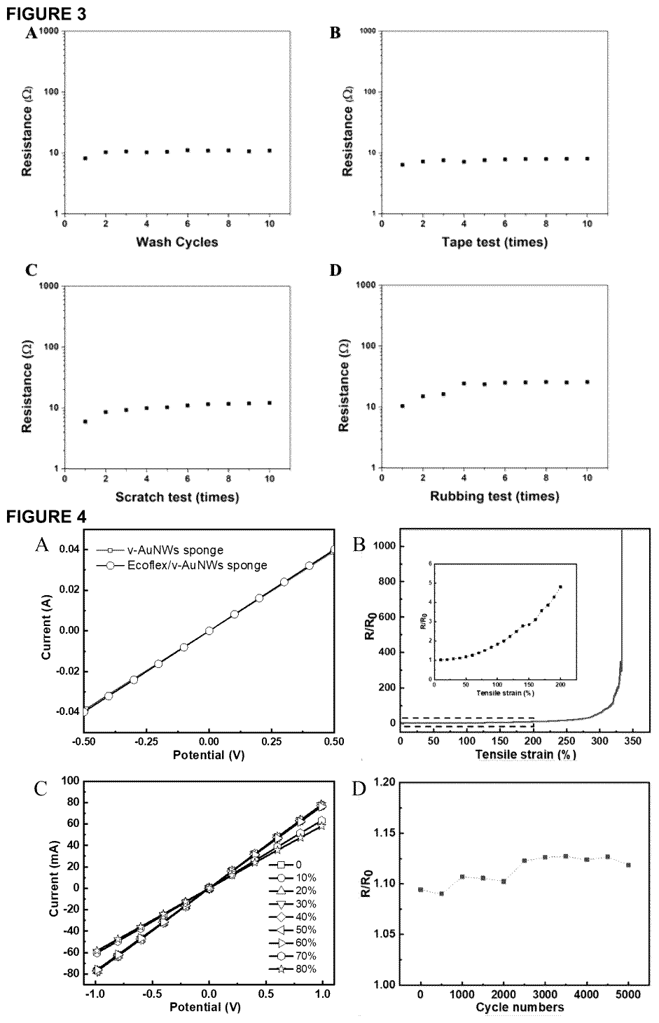

The v-AuNWs also possess ultra-strong adhesion with the PU sponge substrate. The v-AuNWs could survive 10 cycles of the washing test and Scotch tape stripping test without any significant change in resistance (FIGS. 3A and 3B respectively). The strong adhesion is likely due to the use of complexing moieties covalently bonded to the 3D porous elastomeric substrate serving as a bifunctional molecular glue. The amine moiety of APTMS strongly interacts with v-AuNWs, and the silane moiety covalently bonds to the PU sponge substrate. This is also demonstrated by the minimal changes in conductivity of v-AuNWs sponges under harsh fingernail scratching tests and finger rubbing tests over 10 cycles each (FIGS. 3C and 3D respectively). Because of uniform and conformal v-AuNWs coating throughout PU substrate, the gold sponge exhibits an excellent conductivity of 1500 S m⁻¹.

### Strain-Insensitive Deformable Porous Elastic Conductors on 3D Porous Elastomeric Substrates Embedded in Solid Elastomeric Materials

In one embodiment, the present invention provides a highly strain insensitive deformable elastic conductor on a 3D porous elastomeric substrate embedded in a solid elastomeric material, such as, but not limited to, for example, an addition cure silicone rubber such as Ecoflex. The skilled addressee will understand that any solid elastomeric material derived from liquid precursors may be used in accordance with the present invention as a material for embedment or encapsulation of the deformable elastic conductors comprising v-MNWs grown on 3D porous elastomeric substrates.

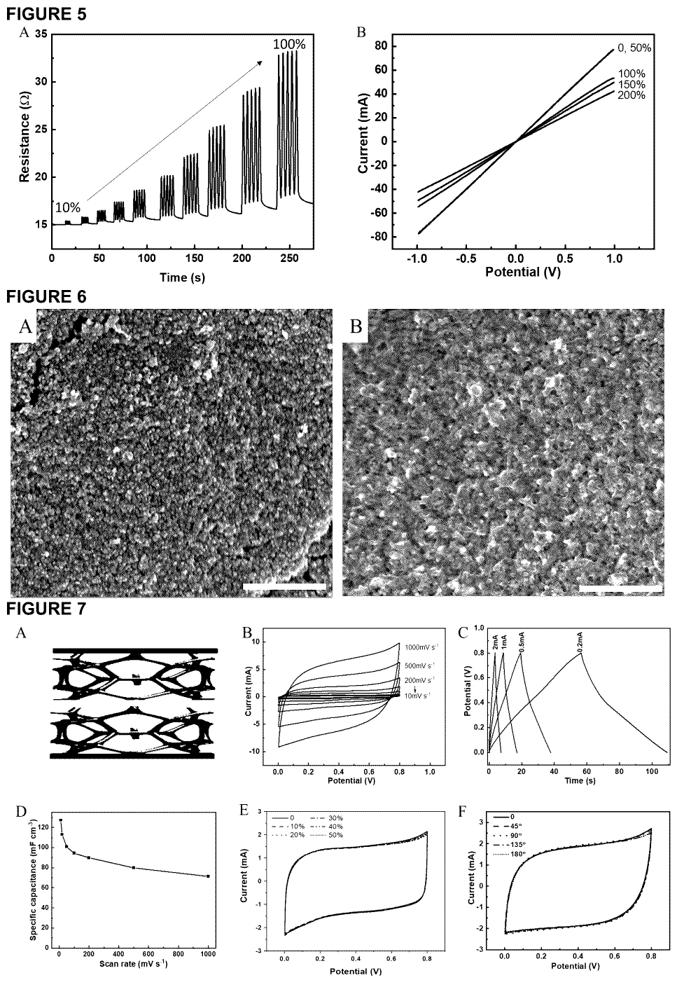

With embedment of the deformable porous elastic conductor of the present invention into a solid elastomeric material (such as Ecoflex), the present invention provides a highly stretchable deformable porous elastic conductor that could stretch up to ˜340% with strain-insensitive conductive properties. Ecoflex embedment did not affect the conductivity of the v-AuNWs on PU sponge substrate as demonstrated by the overlapping I-V curves (FIG. 4A). Remarkably, the embedded v-AuNWs PU sponge displayed outstanding strain-insensitive conductivity. As shown in FIG. 4B, the relative resistance (R/Rₒ) is only 1.26 under 60% tensile strain and 1.83 under 100% tensile strain. Even under 100% tensile strain, the resistance remained low with a value of ˜33Ω (FIG. 5A).

Under a strain less than 50%, all the I-V curves overlap (FIG. 4C), indicating the absence of PU substrate damage. It appears that some substrate started to break over 60% strain, leading to about 0.2% current reduction in comparison to the original unstretched state. With increased strain to 100%, the current continued to drop about 28% (FIG. 5B). With further increased strain to 340%, the conductivity was completely lost (FIG. 4B). This stretchability limit outperforms those sponge-based conductors previously reported in the literature.^{9, 52-54}

The solid elastomer embedded strain-insensitive conductors are highly durable with less than 12% changes in electrical resistance under 5000 stretch-release cycles at 30% strain (FIG. 4D).

### All-Solid-State Soft Supercapacitors

In one embodiment, the present disclosure provides soft supercapacitors comprising the deformable porous elastic conductors of the invention.

The deformable porous elastic v-MNWs conductors of the present invention possess a high voidage of ˜50.8% with interconnected porous structures, which ensure an even distribution of electrolytes throughout pores with intimate contact with the v-MNWs enabling efficient charge transfer.

To enhance the capacitance, in an illustrative embodiment of a soft supercapacitor the inventors deposited a layer of pseudocapacitive polyaniline (PANI) on a v-AuNWs PU sponge by electrodeposition. This led to a slightly rougher surface of the v-AuNWs PU sponge (FIG. 6 A,B). Based on the thus produced PANI/v-AuNWs PU sponge, soft and compressible supercapacitors were assembled (FIG. 7A). Gold sputtered poly(ethyleneterephthalate) (PET) film substrates were used as current collectors. Filter paper soaked in electrolyte served as a separator between the two layers of PANI/v-AuNWs PU sponge and the electrolyte was poly(vinyl alcohol)/H₂SO₄ (PVA/H₂SO₄).

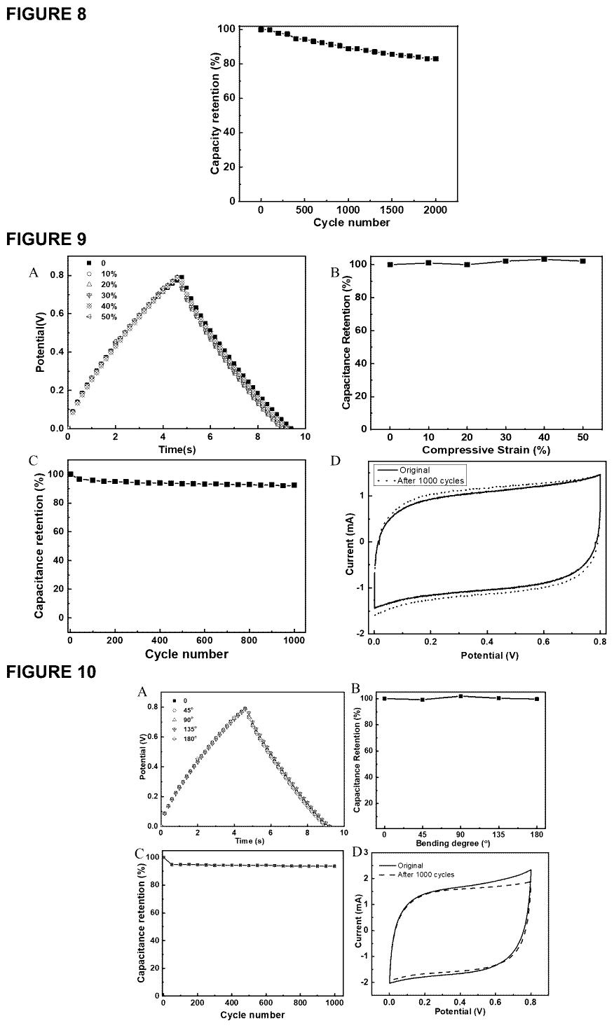

Cyclic voltammetry (CV) and galvanostatic charge-discharge (GCD) tests were carried out with the voltage set between 0 and 0.8 V. As illustrated in FIG. 9B, the CV curves maintain quasi-rectangular shapes with scan rate increased up to 1000 mV s⁻¹. The GCD curves (FIG. 7C) are symmetric triangular shapes indicating reversible charge-discharge behaviour. The volumetric capacitance calculated from CV curves with different scan rates are summarized in FIG. 9D. The PANI/v-AuNWs PU sponge-based supercapacitor exhibited a volumetric capacitance of 127.3 mF cm⁻³ at the scan rate of 10 mV s⁻¹, with a capacitance retention of 70.1% under 200 mV s⁻¹. Next, the stability of the supercapacitors under 2000 cycles of CV tests at 200 mV s⁻¹ was examined (FIG. 8), showing a 83% capacitance retention after 2000 cycles.

The as-prepared PANI/v-AuNWs PU sponge-based supercapacitor could sustain extreme compressing and bending while maintaining its electrochemical performance. As shown in FIG. 7E, when different compressing strains up to 50% are applied to the supercapacitor, there are almost no deviations observed in the CV curves, indicating the excellent mechanical stability of the supercapacitors. The GCD curves shown in FIG. 9A are consistent with the CV curves. There are only very slight differences of capacitance observed under different compression strains, with a capacitance retention of 102% under 50% compression strain (FIG. 9B). In addition, the supercapacitor could retain around 93% of its original capacitance under repeated compression-release cycles after 1000 cycles at 50% strain (FIGS. 9C,D). Consistent results were obtained for the bending tests, where both CV curves (FIG. 7F) and GCD curves (FIG. 10A) remained almost unchanged even when the supercapacitor was bent from 0° to 1800. The capacitance retention is about 99% even under angles of 180° bending strain (FIG. 10B). Even after 1000 bending cycles at an angle of 180°, the supercapacitor still showed a 94% capacitance retention (FIG. 10C, D). Overall, the soft supercapacitors of the present invention outperformed recent literature reports with other materials when being severely deformed.^{13,55}

### Electrocatalysts

In one embodiment, the present disclosure provides for the use of the deformable porous elastic conductors of the invention for applications in catalysis.

In addition to applications in soft electronics, the present inventors have found that the v-MNWs conductors of the present invention also serve as effective 3D catalysts. As an illustrative embodiment, the catalytic activity of the v-AuNWs PU sponge in the reduction of 4-nitrophenol to 4-aminophenol with NaBH₄ was investigated.

The inventors immersed the untreated PU sponge and v-AuNWs PU sponge into the yellow solutions of 4-nitrophenol and NaBH₄. After 20 min, the solution in contact with the v-AuNWs PU sponge turns colourless indicating conversion of 4-nitrophenol to 4-aminophenol. In contrast, the solution contact with untreated PU sponge remains yellow even after several days. The catalytic reaction progress was carefully monitored by UV-vis spectrometry. As demonstrated in FIG. 11B, as the reaction progressed, the characteristic absorption peak of 4-nitrophenol at 400 nm decreased and simultaneously a new peak at 295 nm due to absorption by the 4-aminophenol reaction product appeared and gradually became enhanced. This demonstrates the reduction of 4-nitrophenol to 4-aminophenol.

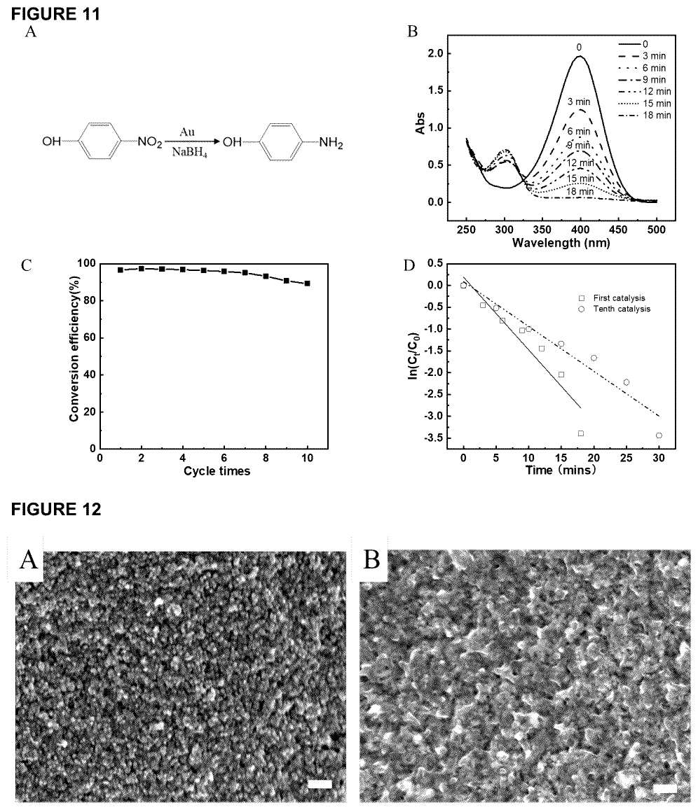

The catalytic reactions did not alter the surface morphologies of the v-AuNWs PU sponge (FIG. 12), which may be attributed to the strong adhesion of the v-AuNWs to the PU sponge substrate and the chemically inert nature of gold.

One advantage of the v-AuNWs PU sponges for catalysis is that they could be reused simply by repeated immersion into a fresh mixed solution of 4-nitrophenol and NaBH₄ without cleaning or any other regeneration process. FIG. 11C shows that the v-AuNWs PU sponge maintained similar catalytic performance, with only a slight decrease (˜10%) in the conversion efficiency even after 10 reaction cycles.

The reaction rate constant K could be estimated via linear regression between In(Cₜ/C₀) (where Cₜ is the concentration of 4-nitrophenol at time t, wherein Cₜ/C₀ values of 4-nitrophenol were measured via the relative intensities of the respective absorbances Aₜ/A₀) and reaction time (t) (FIG. 11D). The estimated reaction constants for the first and tenth cycle are 0.167 min⁻¹ and 0.103 min⁻¹, respectively.

The skilled addressee will understand that the catalytic properties of the deformable porous elastic conductors of the invention may be employed in the catalysis of numerous other chemical reactions apart from the above described exemplary reduction process.

### Soft Battery-Free Wireless Pressure Sensing Antennae

In one embodiment, the disclosure herein provides for the use of the deformable porous elastic conductors of the invention for applications as antennae, including for use in applications in wireless pressure sensing.

The present inventors have found that the v-MNWs conductors of the present invention may also be applied in the implementation of antennae. As an illustrative embodiment, the application of the v-AuNWs PU to a soft, battery-free wireless pressure sensing antenna was investigated.

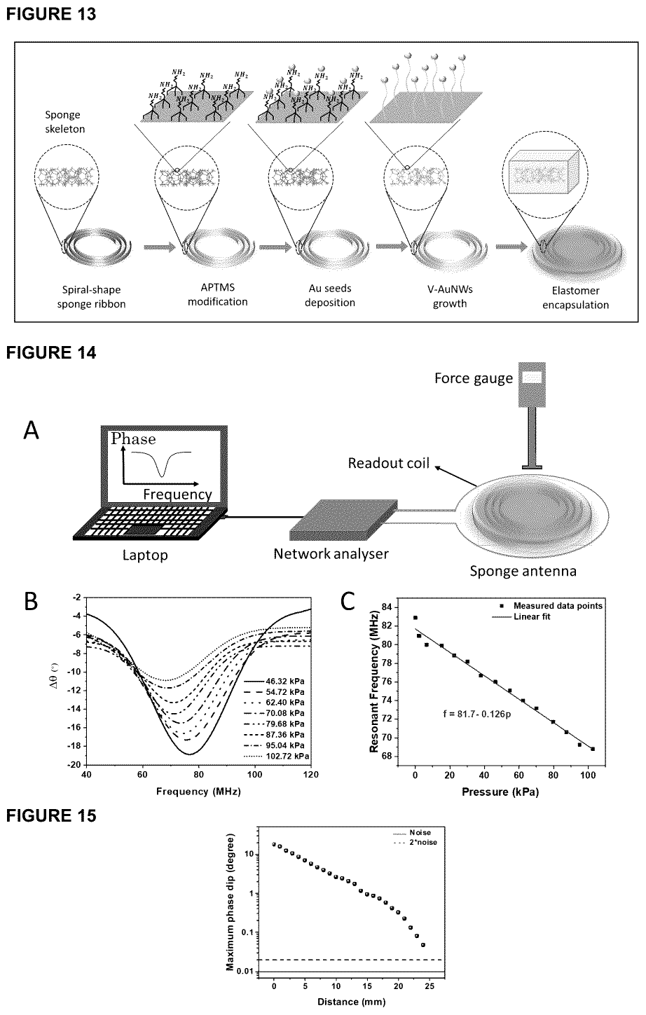

FIG. 13 illustrates the fabrication process of v-AuNWs PU sponge antenna. A piece of PU sponge was first cut into a spiral shape ribbon, upon which v-AuNWs were grown via the method of the invention, as discussed above in relation to FIG. 1.

To test the antenna, the experimental setup is illustrated in FIG. 14A. A vector network analyzer (miniVNA Tiny+, Xuanli Electronic Technology Factory Store) was used to evaluate the antenna and the corresponding phase-frequency spectrum was automatically processed and visualised in the laptop connected to the network analyzer via an USB cable. To achieve inductive coupling with the sponge antenna, the analyzer connects to a two-turn hand-wound copper coil with a diameter of 39 mm and the enamelled copper wire has a diameter of 0.8 mm. Although the maximum detectable distance between the reader coil and sponge antenna is 24 mm (FIG. 15), the detection distance in the following tests are all 2 mm to ensure a consistent and strong signal quality. In addition, a force gauge was placed above the antenna to apply and record external pressure. The applied pressure changes the antenna dimensions, which leads to the changes of capacitance between adjacent sponge ribbons (Cₛ) and inductance of whole spiral inductor (Lₛ). Consequently, the resonant frequency (fₛ) of the sponge antenna varies with varying externally applied pressure.

The resonance frequency of the antenna can be determined by the min-phase method⁵⁶, in which the frequency (fₘᵢₙ) at the minimum impedance phase of the detection coil is regarded as an approximate value of fₛ. The relationship between fₘᵢₙ and fₛ is;

$${f}_{min} = ( 1 + \frac{{k}^{2}}{4} + \frac{1}{8 {Q}^{2}} ) {f}_{s} \qquad ( 1 )$$

where k is the coupling coefficient for characterizing the interaction efficiency between the reader coil and the sponge antenna, which has a value in the range of 0 and 1;⁵⁶ Q is the antenna's quality factor, which is the ratio of fₘᵢₙ and the −3 dB bandwidth in the phase-frequency spectrum. Based on our sponge antennae, Q has a typical value of >5. Therefore, the second and third terms in the bracket approach zero. Thus, the equation that fₘᵢₙ≈f0 can be used for the analysis of gold sponge antenna.

To enhance the antenna's robustness, the spiral-shaped antenna was embedded into Ecoflex, the performance of which is shown in FIG. 14b-c. FIG. 14b clearly shows that fₛ shifts to left and the maximum phase dip monotonically decreases from 18.9 dB to 10.9 dB when the external pressure (p) increases from 46.3 kPa to 102.7 kPa. This indicates that increasing pressure simultaneously decreases the resonant frequency and the signal strength of the antenna in the medium pressure range (10-100 kPa). In addition, FIG. 14c reveals that the resonant frequency response is linear in the pressure range of 0-102.7 kPa with a sensitivity (6, defined as dfₛ/dp) of −126 kHz/kPa. The linear fit of frequency (MHz) versus pressure (kPa) is given by

$${f}_{s} = 81.7 - 0.1 \; 26 p ( R 2 = 98.9 \% ) \qquad ( 2 )$$

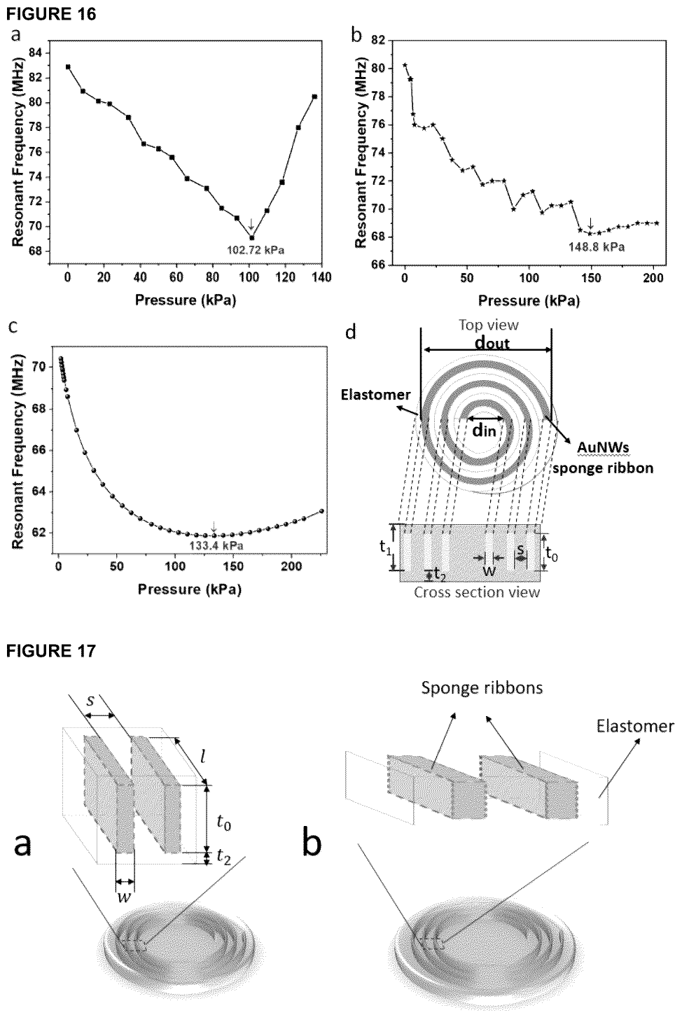

To investigate what happens in the higher pressure region, the inventors kept increasing the external pressure to 140 kPa. The inventors found that when the applied pressure is increased beyond 102.7 kPa, the resonant frequency starts to increase (FIG. 16a). Therefore, 102.7 kPa is regarded as a performance transition point for the v-AuNWs PU sponge antenna embedded in Ecoflex. The skilled addressee will understand that by using different elastomers, other than Ecoflex, and having different elastomeric properties, different performance transition points will be observed.

To further understand this transition point, a computational analysis was conducted for the antenna's performance including simulation and theoretical analysis. Materials inherently tend to expand in directions perpendicular to the direction of compression, in accordance with the Poisson effect⁵⁷. Thus, when the v-AuNWs PU sponge antenna is under pressure, the thickness of the sponge ribbon (t₀) and elastomer (t₂) would decrease, nevertheless the spacing (s) between adjacent sponge ribbons would increase because the elastomer between the ribbons would expand in lateral directions; the length (l) and width (w) of the ribbons would also increase because the ribbons were fully embedded into the elastomer and the elastomer's lateral expansion forced the ribbon to expand simultaneously (FIG. 17). Since the mechanical strength of the v-AuNWs PU sponge antenna is mainly provided by the embedded elastomer, the assumption is made that the Poisson's ratio of sponge antenna equals that of Ecoflex (0.5). Then after measuring the changes of t₀ and t₂, the inventors can calculate the changes of s, l, w following the rules of the Poisson effect.

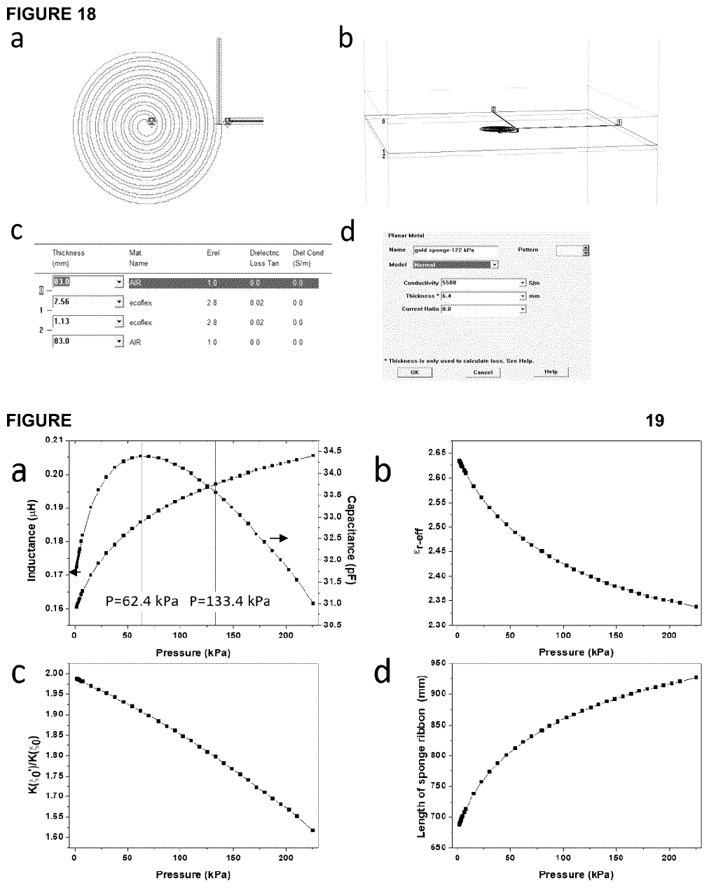

With these data, the present inventors modelled and simulated the antenna's performance in SONNET (FIG. 18). As shown in FIG. 16b, the linearity and simulated frequency-pressure curve is not in agreement with experimental result and the simulated transition point is also different. The reason for this could be that the spacing between adjacent sponge ribbon sections is uneven, which is not consistent with the model. To understand the relationship between resonant frequency and applied pressure, theoretical analysis was further undertaken for the sponge antenna based on the geometrical parameters shown in FIG. 16d.

The sponge antenna's resonant frequency fs is defined by:

$${f}_{s} = \frac{1}{2 π \sqrt{{L}_{s} {C}_{s}}} \qquad ( 3 )$$

The antenna's inductance Lₛ can be described by the following formula⁵⁸:

$${L}_{s} = \frac{{μ}_{0} {μ}_{r} {N}^{2} {d}_{avg}}{2} ( ln ( \frac{2.46}{ρ} ) + 0 \text{.2} * {ρ}^{2} ) \qquad ( 4 )$$

where μ₀=4π×10⁻⁷ H/m, μᵣ is the relative permeability, N is the turns of the sponge ribbon,

d_{avg}=0.5(dₒᵤₜ+dᵢₙ), ρ=(dₒᵤₜ−dᵢₙ)/(dₒᵤₜ+dᵢₙ), dₒᵤₜ is the outer diameter and di, is the inner diameter of the antenna. In equation 4, μ₀, μᵣ and N are constants independent on external pressure. However, d_{avg} and ρ can be directly affected by s and w, which are actually affected by pressure due to the Poisson effect (FIG. 17). Therefore, the relationship between Lₛ and pressure can be obtained after confirming the relationship between d_{avg}, ρ and pressure. As shown in FIG. 19a, Lₛ kept slowly increasing when the applied pressure increased.

The capacitance Cₛ can be written as³⁵:

$${C}_{s} = l * {ε}_{r - eff} * {ε}_{0} * \frac{K ( {ξ}_{0} ′ )}{K ( {ξ}_{0} )} \qquad ( 5 )$$

where l is the sponge ribbon length, ε_{r-eff} is equivalent relative dielectric, ε₀ is the vacuum permittivity, ξᵢ is related to thickness (t₀, t₁, t₂), ξ′ᵢ=\sqrt{1−ξᵢ²}, the value for i is 0, 1 and 2. K(ξᵢ) is the complete elliptic integral of the first kind. Pressure affected l, s, w and t, which could further influence ε_{r-eff} (FIG. 19b) and K(ξ₀′)/K(ξ₀) (FIG. 19c). After quantifying the relationship between ε_{r-eff}, K(ξ₀′)/K(ξ₀) and pressure, we could obtain the relationship between Cₛ and pressure (FIG. 19a).

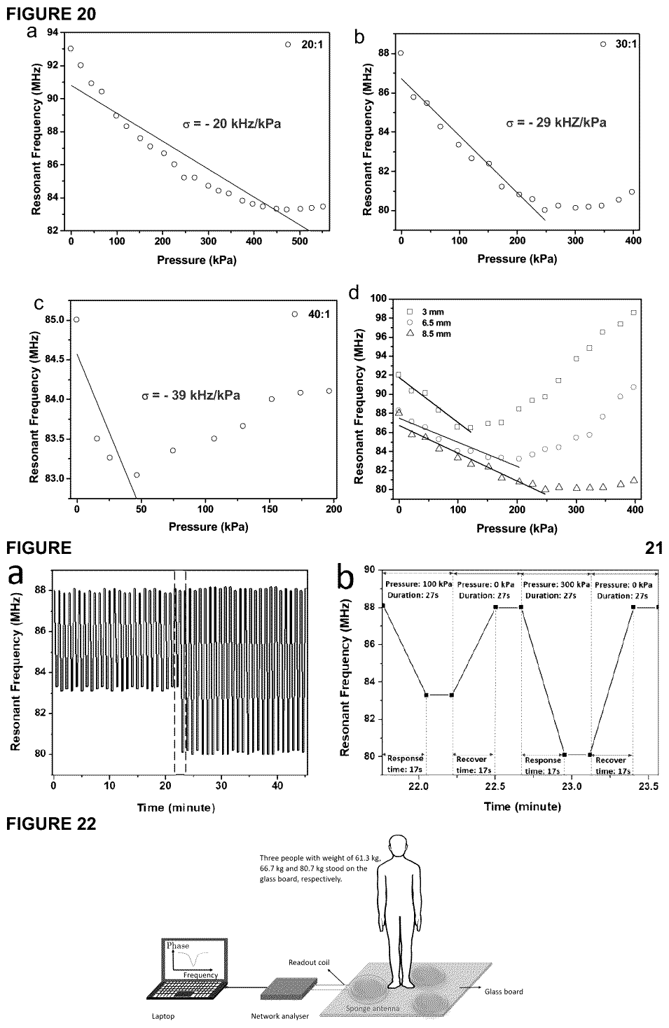

Through above analysis, we could confirm the relationship between Lₛ, Cₛ and pressure. Then we put Lₛ and Cₛ into equation 3 to predict the trend of resonance frequency fₛ. As shown in FIG. 16c, the fₛ decreases first (0-133.4 kPa) and then monotonously increases. As shown in FIG. 19a, when the pressure increased, Lₛ maintained increasing while Cₛ increased first and then rapidly decreased. In detail, when pressure increased from 0 to ˜60 kPa, both Lₛ and Cₛ kept increasing, which led to rapid decrease of fₛ; when pressure kept increasing until ˜130 kPa, capacitance maintained decreasing, which gradually offsets the increase tendency of inductance and finally stops fₛ's decrease at the transition point; beyond this point, the capacitance effect dominates and fₛ kept increasing. Therefore, the calculated transition point is around 130 kPa, which is close to the simulated value and the experimental value. In addition, we found the linearity of the theoretical fₛ—pressure curve is poor compared to experimental result, which might be due to the inconsistency between theoretical and experimental sponge ribbon's length. Specifically, when the sponge antenna is under larger magnitudes of pressure, the ribbon-Ecoflex can't elongate as much as pure Ecoflex, which is attributed to the ribbon's weaker elasticity. To figure out if mechanical property and dimension can adjust the transition point, the v-AuNWs PU sponge antennas with different rigidity of encapsulation elastomer and v-AuNWs PU sponge ribbon thicknesses were respectively tested and compared. At first, three v-AuNWs PU sponge ribbons were embedded into PDMS with different elasticities. The three sponge antennae had similar dimensions and conductivity. To alter PDMS's elasticity, the mixing ratio (w/w) of PDMS base and curing agent was adjusted from 20:1, 30:1 to 40:1. In general, when the mixing ratio between base and curing agent is larger, PDMS is softer. The response of resonant frequency as a function of applied pressure is shown in FIGS. 20a-c. The transition points of the three antennas are respectively 472.5 kPa, 248.0 kPa and 47.0 kPa, which indicates that stiffer PDMS can lead to larger transition point. This makes sense because less pressure is required to achieve the same level of strain for softer PDMS than that for a stiffer one. Thus, the capacitance in a softer v-AuNWs PU sponge antenna can start to dominate at smaller pressure values compared to more rigid samples, which means the resonant frequency can start to increase when smaller pressure value is reached.

In order to study the effect of elastomer rigidity on device sensitivity, the performance of three antennas was linearly fitted (FIGS. 20a-c). The reason for poor linearity shown in FIG. 20c may be that the thickness change of 40:1 PDMS is too dramatic. At the beginning, small pressures (16 kPa) can result in huge thickness variations (4.70 mm), which lead to large elastic resistances for further increasing pressures. For example, though the pressure is increased from 16 kPa to ten times, the thickness change is only 2.72 mm. It is noted that the sensitivity a for three antennas are −20 kHz/kPa, −29 kHz/kPa and −39 kHz/kPa, indicating that softer sponge antennae are more sensitive to applied pressure, which can be attributed to smaller elastic resistances during compression compared to more rigid samples. Accordingly, both the transition point's position and antenna sensitivity are tunable by adjusting the rigidity of embedment elastomer.

The influence of the sponge ribbon thickness, to on the transition pressure and sensitivity of the pressure sensors was also independently investigated. A 30:1 PDMS was used for encapsulation to ensure the consistent rigidity of supporting matrix in all sensors prepared. The ribbon thickness to was adjusted from 3 mm to 8.5 mm. As shown in FIG. 20d, the transition points of sensors with to from 3 mm to 6.5 mm and 8.5 mm are 122 kPa, 204 kPa and 248 kPa, which indicates that the thickness of sponge ribbon can also alter the value of the transition pressure. Furthermore, it was confirmed that larger to can delay the occurrence of the transition point. The reason may be that increased to can increase capacitance but will not affect inductance since conductive trace thickness minimally affects spiral inductance⁵⁸. Specifically, the inductance of the three sensors would be the same if they were under the same pressure. Meanwhile, when to is larger, the capacitance decrease effect on fₛ is smaller and the resonant frequency is more likely to maintain a trend of decreasing with increasing pressure. The overall effect is that a larger to leads to a higher transition point.

To demonstrate the performance reliability of the v-AuNWs PU sponge antenna, durability tests were conducted for the antenna encapsulated in PDMS (FIG. 21). The mixing ratio of PDMS base and curing agent was 30:1 for the embedment elastomer, and the sponge thickness was 8.5 mm. 100 kPa and 300 kPa pressure were respectively applied to the antenna for 25 cycles. During every cycle, static pressure was applied to the antenna for 27 s and then removed for 27 s. Within the 27 s periods, the first 17 s is the network analyser response time and the subsequent 10 s is for obtaining a stable resonant frequency (RF) signal from the antenna. Based on the analysis of the periodic resonant frequency curves, the inventors found that the sensor could maintain stable output under pressure and recover to its initial state when loads are released. For example, when 100 kPa pressure was applied, the detected RF values were around 83.2 MHz with a fluctuation of ±0.1 MHz. After the removal of applied pressure, the RF signal returned to its original value (88.05±0.15 MHz). These results suggest excellent mechanical robustness and good reproducibility of the v-AuNWs PU sponge antenna.

The versatility of the v-AuNWs PU sponge antenna sensors in tuning sensitivity and pressure sensing ranges and their high durability indicate the numerous potential applications in soft electronics^{1, 59, 60} As a proof of concept, the use of the encapsulated v-AuNWs PU sponge antenna as a soft battery-free balance for measuring body weight in both static and dynamic conditions was demonstrated.

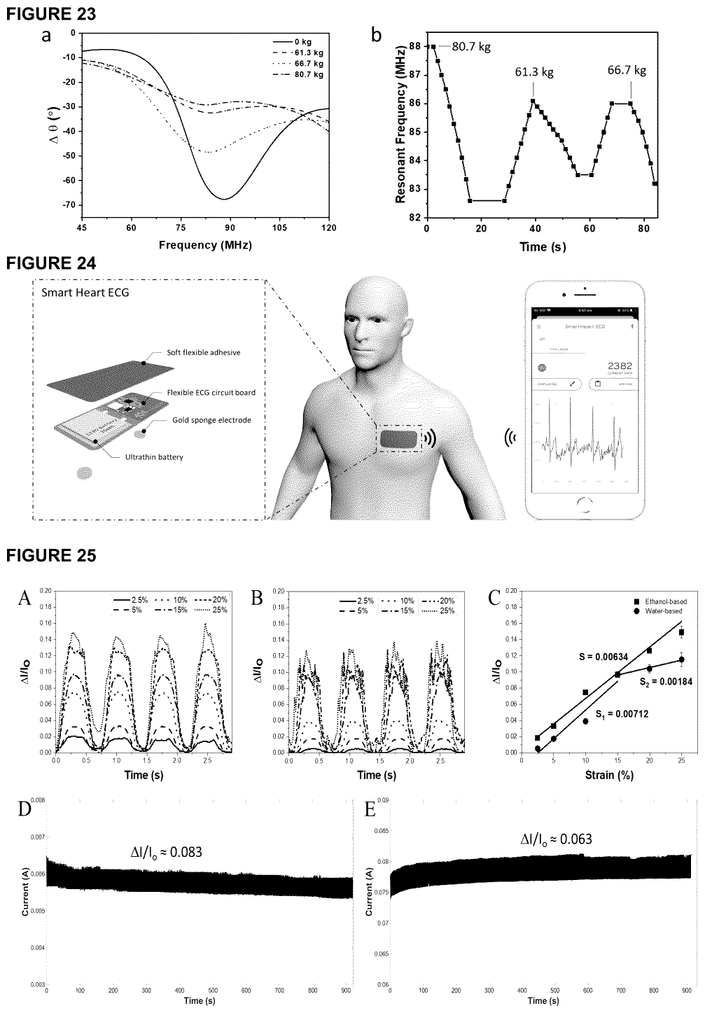

The v-AuNWs PU sponge antenna used in this application was encapsulated in PDMS with a sponge ribbon thickness of 8.5 mm and the mixing ratio of PDMS to curing agent was 30:1. As shown in FIG. 22, three sponge antennas with the same area and rigidity were placed under a glass board (5 kg) and the reader coil was placed under the leftmost antenna. Three volunteer subjects with weights of 61.3 kg, 66.7 kg and 80.7 kg stood on the board respectively. The pressures applied to the leftmost antenna were 101.98 kPa, 110.29 kPa and 131.8 kPa. The resonant frequencies of three phase dip-pressure curves were 83.7 MHz, 83.2 MHz, and 82.6 MHz (FIG. 23a).

Putting these values into the linearity function in FIG. 20b (fₛ=86.7-0.02914p), the inventors can obtain the corresponding pressure values, 104.7 kPa, 118.7 kPa, 139.7 kPa. These results are in very close agreement to the experimental results, which indicates that the v-AuNWs PU sponge antenna is suitable for distinguishing body weight in static situations. Then the three volunteer subjects stood on the glass board one by one to test the function of weighing people's weight in dynamic situations.

The first volunteer subject with weight of 80.7 kg stood on the glass board from 2.3 s to 27.9 s. As shown in FIG. 23b, the response time for the network analyser is 13.5 s. During this period, the impedance phase-frequency curve kept shifting to the left, indicating that the resonant frequency was decreasing. After the curve became static, the fₛ kept the value of 82.6 MHz until 27.9 s. From 27.9 s to 37.2 s, the antenna started to recover. Before it fully recovered, the second volunteer subject with weight of 61.3 kg stood on the glass board and the f started to decrease again and finally became steady at 83.5 MHz until 60.5 s. Finally, the third volunteer subject with weight of 66.7 kg stood on the glass board at 67 s. Then the curve remained static from 68 s to 76.3 s, which may be due to the offset of recovering tendency and left shifting tendency for fₛ. Subsequently, the curve started to shift to the left and remained static at 83.8 s. The resonant frequency maintained 83.2 MHz until the end of the trial. It is noted that the final stabilized frequencies (82.6, 83.2 and 83.5 MHz) are related to the volunteer subject's weights and this relationship is also in agreement with the calibration curve shown in FIG. 20b. Therefore, v-AuNWs PU sponge antenna is capable of distinguishing people's weight under both static and dynamic situations.

Surprisingly and advantageously, the linear detection range of optimally designed sensors incorporating the v-AuNWs PU sponge antennae in accordance with the present invention by far surpasses that of many existing wireless pressure sensing technologies, as summarised in Table 1:

TABLE 1
comparison of linear detection range of existing
wireless pressure sensing technologies

| | Sensing | Linear detection |
|---|---|---|
| Reference | mechanism | range (kPa) |
| 37 | Capacitive | 0-26.7 |
| 36 | Capacitive | 0-6.7 |
| 40 | Capacitive | 0-24 |
| 32 | Capacitive | 0-26.7 |
| 29 | Inductive | −1.3-2 |
| 61 | Inductive | 0-6.7 |
| 62 | Inductive | 0-8 |
| 28 | Inductive | 0-20 |
| 39 | Inductive-capacitive | 0-4 |
| 37 | Inductive-capacitive | 0-6.67 |
| Present Invention | Inductive-capacitive | 0-248 |

The v-AuNWs PU sponge sensors of the present invention can be operated wirelessly without a power supply. Moreover, they possess finely and widely adjustable linearly responsive pressure ranges and sensitivities, which provide potential for a wide range of applications including robotics and in health care, for example pressure mapping of diabetic's feet, bed matrix of aged care patients, etc.

### Wearable Soft Electrophysiological Sensing Devices

In some embodiments, the v-MNWs deformable porous elastic conductor of the present invention may be utilised as a dry soft electrode for implementation in data collection devices, including but not limited to, for example, Electrocardiograph (ECG) devices, or Electromyograph (EMG) devices, or Electroencephalograph (EEG) devices. In some such embodiments, the device is wearable. In some such embodiments, the device is capable of wirelessly transmitting data to a separate data logging and processing device.

In an exemplary implementation of such a device, FIG. 24 depicts a schematic of a wearable ECG device. The wearable ECG is a thin and flexible ultrathin wearable ECG module with dry v-AuNWs PU sponge electrodes, which may be worn on a patient's or subject's chest to continuously monitor heart electrical activity, to calculate the precise real-time heart rate and to detect any heartbeat abnormalities. The wearable ECG is capable of transmitting data wirelessly via a Bluetooth Low Energy (BLE) module to a separate data logging and processing device such as a smartphone/tablet device or computer.

The wearable ECG monitoring module incorporates a miniaturized flexible single lead ECG module paired with 1 mm ultrathin battery. The wearable ECG monitoring module has a size of 6.1 cm×2.6 cm and thickness of 4 mm including the dry v-AuNWs PU sponge electrode and soft flexible sealing bandage layer.

With reference to FIG. 24, the ECG electrode used in the wearable ECG monitoring module is a bio-compatible deformable dry electrode comprising gold nanowires grown uniformly throughout a PU sponge substrate. The thickness of the electrode is approximately 1.5 mm, which is much thinner and more comfortable than electrodes currently available on the market which typically have a thickness of 2-3 mm. The wearable ECG module is a reusable module with a dry v-AuNWs PU sponge electrode that can be cleaned and sanitized using alcohols.

The wearable ECG device may be adhered to the user's chest shown in FIG. 24. The data collected from the device is wirelessly transmitted via an integrated Bluetooth Low Energy module to a data-logging and processing application on a smartphone/tablet device or computer. This allows the user to wirelessly collect the ECG signal data with extreme comfort and without causing any burden to the user.

The fabrication method of the v-AuNWs PU sponge electrode was tuned to achieve optimum strain and compression insensitivity to conductivity. To do this, a pre-treated PU sponge is soaked in (3-Aminopropyl)triethoxysilane in ethanol and another pre-treated PU sponge is soaked in (3-Aminopropyl)triethoxysilane in water. After that, the PU sponges are dipped in gold seed solution and then growth solution to grow v-AuNWs. The tensile strain insensitive performance of v-AuNWs grown on PU sponge functionalised with (3-Aminopropyl)triethoxysilane (APTES) in ethanol and APTES in water were plotted in FIGS. 25a and b. FIG. 25c shows the gauge factor of both samples as a function of strain (2.5%-25%). The gauge factor for both samples at low tensile strains of 2.5% to 15% were similar. The v-AuNWs grown on PU sponge functionalised with aqueous APTES has gauge factor of 0.00712 at low tensile strains of 2.5% to 15% and subsequently reduced to gauge factor of 0.00184 at higher tensile strains of 15% to 25%. v-AuNWs grown on PU sponge functionalised with ethanolic APTES has linear gauge factor of 0.00634 over tensile strains of 2.5% to 25%. This shows that the v-AuNWs grown on PU sponge functionalised with aqueous APTES is more tensile strain insensitive than the v-AuNWs grown on PU sponge functionalised with ethanolic APTES. Both samples have consistent performance while undergoing cyclic tensile strains of 10% over 1000 cycles as depicted in FIGS. 25d and e.

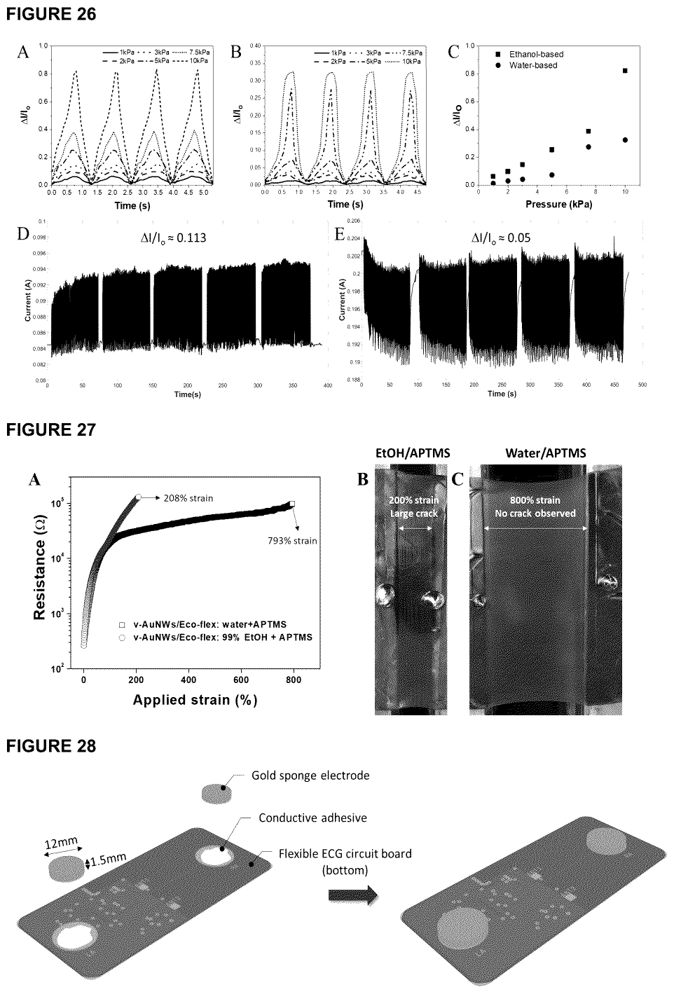

The compressive strain insensitive conductivity performance of v-AuNWs grown on PU sponge functionalised with APTES in ethanol and APTES in water were plotted in FIGS. 26a and b. FIG. 26c shows that the v-AuNWs grown on PU sponge functionalised with aqueous APTES is more insensitive to compressive strain in the range of 1 kPa to 10 kPa compared to v-AuNWs grown on PU sponge functionalised with ethanolic APTES. Both samples have consistent performance while undergoing cyclic compressive strains of 5 kPa over 1000 cycles as depicted in FIGS. 26d and e.

Without wishing to be bound by theory, the inventors believe that these surprising and unexpected improvements in strain insensitive conductivity observed when aqueous functionalising agents are employed to prepare the deformable porous elastic conductors of the invention, are related to a different surface morphology and/or geometry of the covalent attachment of the complexing groups to the porous elastomeric substrate arising from the use of water as solvent.

The growth time of the v-AuNWs on the PU sponge functionalised with ethanolic APTES solution and aqueous APTES solution were also studied. The v-AuNWs grown on the PU sponge functionalised with aqueous APTES reached optimum electrical resistance of ˜2.24Ω after 120 seconds of growth whereas the v-AuNWs grown on the PU sponge functionalised with ethanolic APTES needed 180 s to achieve an electrical resistance of 5.03Ω. The v-AuNWs growth time on both PU sponge samples and their electrical conductivity is summarised in table 2:

TABLE 2
v-AuNWs growth time and electrical conductivity of
v-AuNWs PU sponge (all functionalising agent solutions
were prepared at a concentration of 5 mM APTES)

| Growth time | 30 s | | 60 s | | 90 s | | 120 s | | 150 s | 180 s |
|---|---|---|---|---|---|---|---|---|---|---|
| Water-based | 3.59 | Ω | 2.54 | Ω | 2.43 | Ω | 2.24 | Ω | 2.21 Ω | 2.05 Ω |
| Ethanol-based | 1.37 | kΩ | 146.51 | Ω | 13.68 | Ω | 10.57 | Ω | 7.59 Ω | 5.03 Ω |

The tensile strain insensitive conductivity performance of v-AuNWs grown on a 2D ecoflex stretchable elastomeric substrate functionalised with (3-Aminopropyl)trimethoxysilane (APTMS) in ethanol and APTMS in water were studied and plotted in FIG. 27a. FIG. 27a shows that the v-AuNWs grown on ecoflex substrate functionalised with aqueous APTMS is able to sustain up to 793% of strain while remaining conductive whereas v-AuNWs grown on ecoflex substrate functionalised with ethanolic APTMS lost its conductivity at 208% of tensile strain. FIG. 27b shows that the v-AuNWs grown on ecoflex substrate functionalised with ethanolic APTMS has large cracks at 200% tensile strain whereas for v-AuNWs grown on ecoflex substrate functionalised with aqueous APTMS has no cracks observed even after being stretched to 800%. The v-AuNWs grown on ecoflex functionalised with APTMS in varying amounts of ethanol and water, and their stretchability, sheet resistance and adhesion strength of the nanowires to the substrate is summarised in table 3:

TABLE 3
Summary of stretchability, sheet resistance and adhesion
strength of v-AuNWs growth on ecoflex functionalised
with APTMS diluted with varying amounts of
water and ethanol (all functionalising agent solutions
were prepared at a concentration of 5 mM APTMS)

| H₂O:EtOH | Stretch- | Sheet resistance | |
|---|---|---|---|
| (v:v) | ability | (Ω sq⁻¹) | Tape test |
| 0:100 | 208% | 18.5 | Weak adhesion |
| 10:90 | 343% | 16.5 | Weak adhesion |
| 50:50 | 784% | 13.6 | Strong adhesion |
| 90:10 | 804% | 24.6 | Strong adhesion |
| 100:0 | 793% | 19.5 | Strong adhesion |

The tape test is performed by applying a strip of Scotch Tape™ to the surface of the gold nanowires ecoflex sample, then pulling the tape off the sample. The tape is visually inspected for any removal of nanowires. If large gold patches appear on the tape, the adhesion of the gold nanowires on the ecoflex surface is deemed to be weak and if no large gold patches appear on the tape, the adhesion of the gold nanowires on ecoflex surface is deemed to be strong.

FIG. 28 depicts the method of assembly of the dry v-AuNWs PU sponge electrode on to the flexible ECG circuit board. A disc of sponge was cut into 12 mm in diameter and approximately 1.5 mm thick. A conductive adhesive such as silver paste is applied to the terminal pad on the ECG circuit board, after which the dry v-AuNWs PU sponge electrode is attached onto the area covered with conductive adhesive and then allowed to air dry for 2 hours. The conductivity of the dry v-AuNWs PU sponge electrode and the terminal pad on the circuit board is tested to ensure that it is below 100.

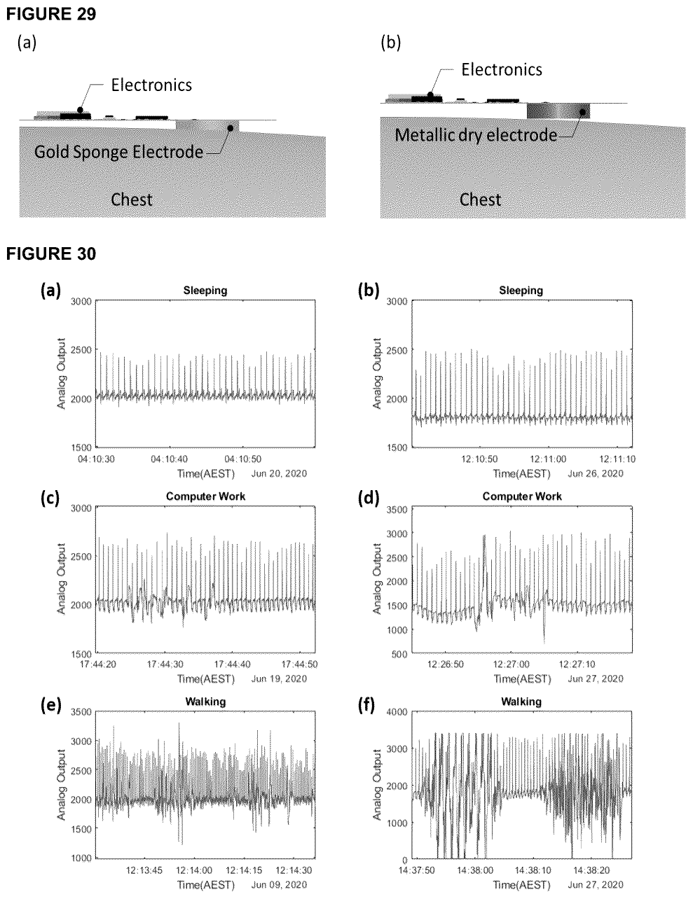

Due to the presence of the electronic parts and battery on the flexible ECG circuit board, the bending of the module is limited. The benefit of using a dry gold nanowire sponge-based electrode with the flexible ECG unit is that its ability to compress allows the electrode to fully contact with the user's chest surface (shown in FIG. 29a), allowing a more stable ECG signal recording and suppressing motion artefacts. On the other hand, the rigid metallic dry electrode is not compressible and will stay afloat when attached to the chest surface as shown in FIG. 29b. This will cause the electrode to glide around the user's chest during movement and the noise from motion artefacts due to unreliable and inadequate skin contact is likely to render the ECG signal collected useless.

To determine the reliability of the dry v-AuNWs PU sponge ECG electrode, a comparison test with the commercial silver/silver chloride (Ag/AgCl) gel-based electrode was performed. The test was carried out by collecting the ECG signal using two different electrodes while the user was sleeping, performing computer work and walking. As shown in FIGS. 30a and b, as there is not much movement during sleeping, the performance of the dry v-AuNWs PU sponge and gel-based ECG electrodes are very similar. However, when the user is performing computer work such as typing and using the mouse, motion artefacts are observed in both the dry v-AuNWs PU sponge and gel-based electrodes as shown in FIGS. 30c and d. The gel electrode has poorer performance than the dry v-AuNWs PU sponge electrode when monitoring cardiac activity during motion. As can be seen at approximately 12:26:56 μm in FIG. 30d, the noise from motion artefacts has fully swamped the ECG signal making this portion of the signal useless.

The performance of the gel electrode deteriorates even further when the user's activity intensified. While walking, the ECG signal collected via the gel electrode is completely distorted as seen in FIG. 30f, and when the user stops walking, the signal returns to normal. In contrast, for the ECG collected using the dry v-AuNWs PU sponge electrode of the present invention, the signal is slightly distorted however the distinctive feature of ECG signal is still identifiable, and the noise produced is not so great as to render the signal unusable. This shows that the dry v-AuNWs PU sponge electrode of the present invention outperformed the existing commercially available Ag/AgCl gel-based electrode when performing ECG monitoring during motion, which is especially useful for use in everyday ECG monitoring and for athletes and during sporting activities.

The advantage of the dry v-AuNWs PU sponge electrode in accordance with the present invention over the currently used gel electrode is its low profile (1.5 mm vs 3 mm) and lightweight that suppresses the moment of inertia thereby reducing the movement of the electrode during human movement. The gel-based electrode, despite ensuring skin contact, has an inherent fluidic feature that causes micro-gliding movements on the skin which also contribute substantially to producing motion artefacts.

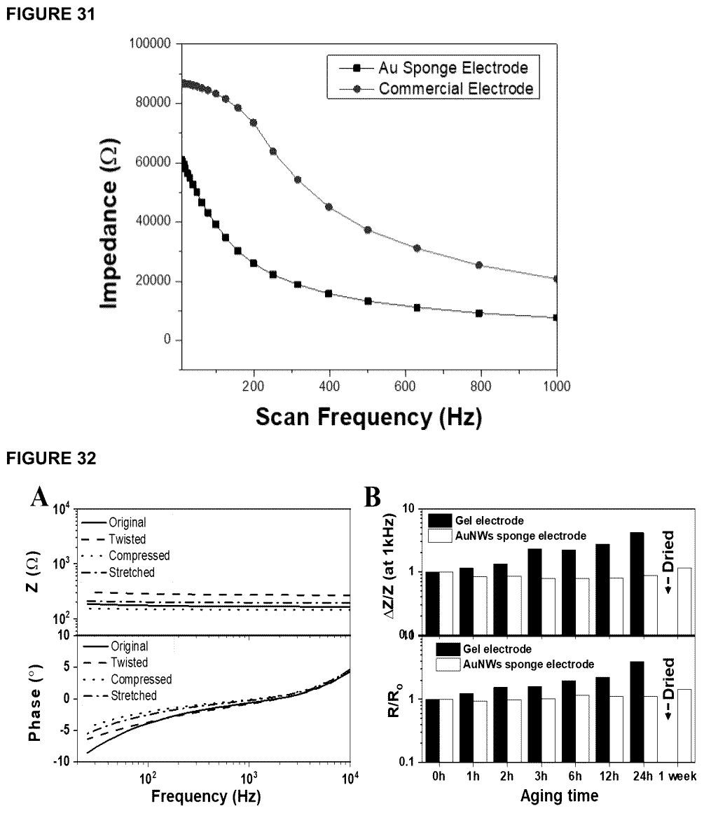

Another shortcoming of the gel-based electrode is its high skin-electrode impedance compared to the v-AuNWs PU sponge electrode. A comparison of the impedance of the electrodes is shown in FIG. 31, where the v-AuNWs PU sponge has impedance that is significantly lower than the commercial gel-based electrode over a scan frequency range of 0 to 1000 Hz. The low skin-electrode impedance v-AuNWs PU sponge electrode contributes to a stable and high amplitude ECG signal collection.

Furthermore, to simulate the environmental conditions likely to be encountered in biomedical or biophysiological sensor applications, the deformation-insensitive impedance of v-AuNWs PU sponge electrodes was evaluated in an artificial sweat solution containing 3 mM ammonium chloride, 22 mM urea, 0.4 mM calcium chloride, 50 μM magnesium chloride, 10 mM potassium chloride, 137 mM sodium chloride, 25 μM uric acid and 100 μM glucose (pH=7). Because the frequency range of the electrophysiological signals is less than 1,000 Hz, the impedance and phase of v-AuNWs PU sponge were compared at the frequency of 1,000 Hz with and without applying a tensile strain of 40%, compressive strain of 80%, and twisting strain of 1080°. The impedance and phase without strain were 175.7±7.6 0 and ˜3.89±0.15°, respectively. This value was slightly shifted to 203.8±37.1 Ω, 127.5±11.4Ω, and 284.8±4.9Ω in response to tensile, compressive, and twisting strain, respectively (FIG. 32A). In ambient conditions, the dry v-AuNWs PU sponge exhibited stable conductivity and impedance, outperforming commercial gel electrodes. As shown in FIG. 32B, the commercial gel ECG electrode showed an obvious increase in its impedance (ΔZ/Z>3) and conductivity (R/R0>3.5) after aging in ambient conditions for 24 h, and then became completely dry and non conductive after 72 h. In contrast, the electrode of the present invention exhibited negligible changes in impedance (ΔZ/Z<1.2) and conductivity (R/R₀<1.3) even after aging for 1 week.

The dry gold v-AuNWs PU sponge ECG electrodes of the present invention provide a much more robust electrode than the conventional Ag/AgCl gel-based electrodes, with a lower impedance than the gel-electrode, being less prone to motion artefacts, being more durable, reusable, sanitisable and having a longer lifespan compared to gel-electrodes.

### Soft Pressure and Strain Sensor

In one embodiment, the disclosure herein provides pressure sensors and strain sensors comprising the deformable porous elastic conductors of the invention.

While the deformable porous elastic conductors of the invention exhibit strain insensitive conductivity, the present inventors have found that the v-AuNWs PU sponges of the present invention possess a degree of useful linearity in their responses to strain when measured as relative change in resistance with strain (ΔR/Rₒ) or relative change in current with strain (ΔI/Iₒ), and may therefore also be applied as soft pressure and strain sensors by tuning the growth of the v-AuNWs on PU sponge. Dry gold v-AuNWs PU sponges were fabricated with growth solutions diluted to 25 vol %, 50 vol % and 75 vol % in ethanol and the mechanical properties of each v-AuNWs PU sponge were investigated.

The electrical resistance of v-AuNWs PU sponges is summarised in Table 4 below:

TABLE 4
Summary of electrical resistance of v-AuNWs PU
sponges grown with diluted growth solutions

| Growth solution | |
|---|---|
| concentration | Electrical resistance (Ω) |
| 100 vol % | 4.29 Ω |
| 75 vol % | 10.9 Ω |
| 50 vol % | 35.2 Ω |
| 25 vol % | 128.90 kΩ |

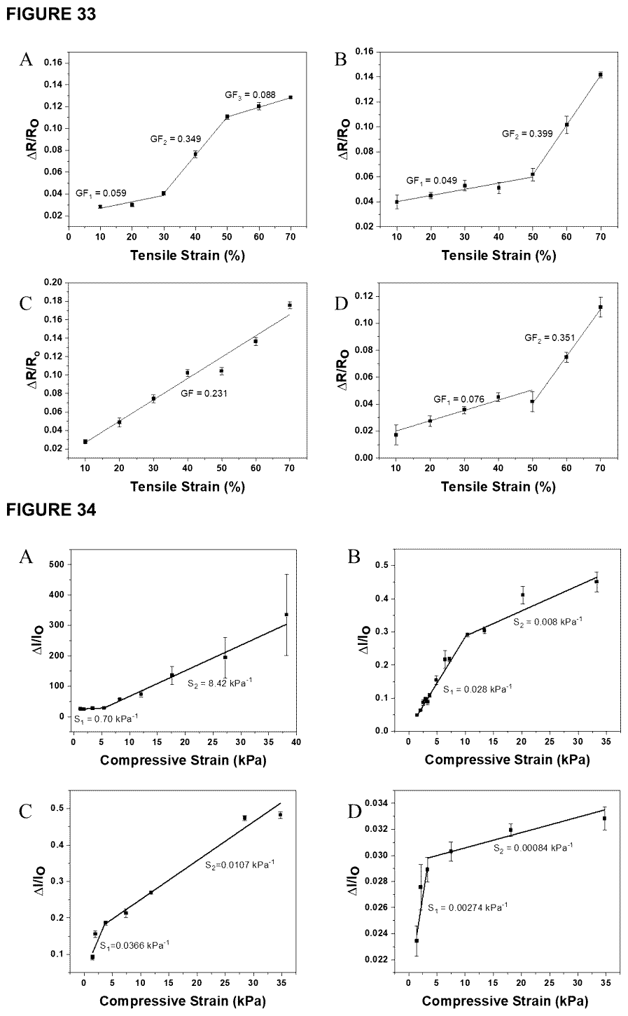

FIG. 33A shows that the v-AuNWs PU sponge grown with 25 vol % diluted growth solution offers a strain sensor with good degree of linearity in terms of changes in relative resistance with strain (ΔR/Rₒ) in the range of 30-50% tensile strain while the V-AuNWs PU sponge grown with 50 vol % and 100 vol % diluted growth solution offers good tensile strain linearity in the 50-70% range (FIGS. 33B and 33D). v-AuNWs PU sponge grown with 75 vol % diluted growth solution provides a useful linear tensile strain response within the 10-70% range (FIG. 33C) which is the most suitable to be used as a tensile strain sensor. It is worth noting that these v-AuNWs PU sponges, unlike the elastomer embedded embodiments of the invention, suffer plastic deformation upon stretching >70%.

The pressure sensing performance of the v-AuNWs PU sponges grown with different vol % of diluted growth solution was also investigated. FIG. 34A shows that v-AuNWs PU sponge grown with 25 vol % of diluted growth solution offers a good linear response to pressure in terms of relative change in current (ΔI/I₀) with compressive strain beyond ˜5 kPa with a sensitivity of 8.42 kPa⁻¹. The other v-AuNWs PU sponges exhibit poor sensitivity as shown in FIG. 34B to 34D. The v-AuNWs PU sponge pressure sensor was compressed completely upon ˜40 kPa of compression.

The foregoing example demonstrates that the performance characteristics and insensitivity or sensitivity to strain of the deformable porous elastic conductors of the present invention may be tuned for suitability to a particular application by employing a method of fabrication wherein the growth of the nanowires is tuned by fabricating a series of the deformable porous elastic conductors with varying concentrations of growth solution.

The skilled addressee will understand that other v-MNWs deformable porous elastic conductors may equally employed in wearable devices in accordance with the above described embodiment. In particular, v-MNWs deformable porous elastic conductors comprising alternative noble metals such as platinum, palladium or rhodium would be especially advantageous as they would confer similarly high conductivities whilst also possessing the advantageous properties of biocompatibility, chemical inertness and resistance to corrosion.

### EXAMPLES

### Materials

Gold (III) chloride trihydrate (HAuCl₄·3H2O, 99.9%), sodium citrate tribasic dehydrate (SC, 99.0%), sodium borohydride (NaBH₄, 99.99%), (3-aminopropyl)trimethoxysilane (APTMS), (3-Aminopropyl)triethoxysilane (APTES), 4-mercaptobenzoic acid (MBA, 90%), L-ascorbic acid (L-AA), poly (vinyl alcohol) (PVA) powder, sulphuric acid (H₂SO₄), 4-nitrophenol (4-NP) and ethanol (analytical grade) were purchased from Sigma-Aldrich. All chemicals were used as received unless otherwise indicated. Polyurethane domestic kitchen sponge was purchased from Coles, Australia or from Advance Imports Pty, Ltd. All solutions were prepared using deionized water (resistivity >18 MO cm⁻¹). Conductive wires were purchased from Adafruit. Ecoflex (0030) was purchased from Smooth-on, Inc. PDMS elastomer base and curing agent (Sylgard 184) were purchased from Dow Corning.

### Characterisation

SEM images were obtained with an FEI Helios Nanolab 600 operated at 5 kV beam voltage.

Bending tests of supercapacitors and stretching tests of conductors were performed using motorized moving stages (THORLABS model LTS150/M) and electrical signals were recorded by a Parstat 2273 electrochemical system (Princeton Applied Research).

Pressure tests were conducted using a computer-based user interface used to apply and record external pressure, and a force sensor (ATI Nano17 force/torque sensor 1/80N resolution without filtering) and a Maxon Brushless DC motor. The return loss and impedance phase signals were directly obtained from Vector Network Analyzer miniVNA Tiny+(Xuanli Electronic Technology Factory Store).

UV-vis spectrometry in the catalysis experiment was detected by Agilent Technologies Cary 60

Conductivity was calculated from the multimeter data of metal sponge samples.

Sheet resistances were measured using a Jandel four-point probe.

### Example 1—Synthesis of Deformable Porous Elastic Conductors on 3D Porous Elastomeric Substrates

Firstly, 3-5 nm seed gold nanoparticles were synthesized. 1 mL, 34 mM sodium citrate was added to a conical flask containing 100 mL H₂O under vigorous stirring. Then 1 mL, 24 mM HAuCl₄ was added into this mixture. After 1 min, 3 mL of ice-cold, freshly prepared 0.1 M NaBH₄ solution was added with stirring. The solution turned brown immediately and gradually changed to a wine red colour. The solution was then stirred for 5 min and stored at 4° C. until needed.

The 3D porous elastomeric substrate (polyurethane sponge) was washed successively in DI water and ethanol three times and then oven dried. The 3D porous elastomeric substrate was cut into desired sizes. To grow vertical gold nanowires on the skeleton of the 3D porous elastomeric substrate, the substrate was pre-treated with 10 min air plasma to render the surfaces of the substrate fully hydrophilic, and enable their functionalisation with complexing moieties.

Next, the substrate was functionalised by soaking in APTMS (5 mM) ethanol solution as functionalising agent for two hours. Optionally, this step may be conducted in the presence of the application of sonication and/or the application of negative pressure to facilitate infiltration or penetration of the functionalising agent into the 3D porous elastomeric substrate.

After washing with ethanol to remove unreacted APTMS and complete drying under a stream of dry N₂ gas, the 3D porous elastomeric substrate was immersed into a seed solution containing a suspension of nanoparticulate Au seeds for another two hours to anchor Au seeds onto the functionalised 3D porous elastomeric substrate, via complexation of the Au seeds to the pendant nitrogen moieties of the APTMS groups covalently bonded to the 3D porous elastomeric substrate in the previous step. Optionally, this step may be conducted in the presence of the application of sonication and/or the application of negative pressure to facilitate infiltration or penetration of the seed solution into the 3D porous elastomeric substrate.

After complexation of the Au metal nanoparticulate seeds to the functionalised 3D porous elastomeric substrate, the substrate was washed with DI water and dried under a stream of dry N₂ gas.

The functionalised 3D porous elastomeric substrate with complexed Au metal nanoparticle head groups was then immersed in a v-AuNWs growth solution of ethanol/water (v/v=1:1.2), which contains HAuCl₄ (12 mM), ligand MBA (1.1 mM), and reducing agent L-AA (30 mM). Optionally, this step may be conducted in the presence of the application of sonication and/or the application of negative pressure to facilitate infiltration or penetration of the growth solution into the 3D porous elastomeric substrate.

After 5 min, the sample was washed in ethanol and under a stream of dry N₂ gas. The deformable porous elastic conductor comprising enokitake-like vertically aligned gold nanowires sponge was thus prepared. The extent of growth of the nanowires may be modulated by adjusting the length of time in which the 3D porous elastomeric substrate with complexed Au metal nanoparticle head groups is exposed to the growth solution.

### Example 2—Fabrication of Strain-Insensitive Deformable Porous Elastic Conductors on 3D Porous Elastomeric Substrates Embedded in Solid Elastomeric Materials

A mixture of Ecoflex curable silicone fluids A and B with a weight ratio of 1:1, was gently poured onto the as-prepared deformable porous elastic conductor of Example 1. Optionally, this step may be conducted in the presence of the application of sonication and/or the application of negative pressure to facilitate infiltration or penetration of the liquid elastomer precursors into the 3D porous elastomeric substrate. Then the composite in pre-cured Ecoflex was degassed in a desiccator for 2 hours, until no gas bubbles were observed on the surface of the mixture. Ecoflex encapsulation was completed by leaving the composite material in an oven at 60° C. for 1 h.

The Ecoflex embedded deformable porous elastic conductor composite samples were then cut into strips to provide strain-insensitive conductors (the volume of the conductor samples was 3 cm×1 cm×0.2 cm).

### Example 3—Fabrication of all-Solid-State Soft Supercapacitors

Firstly, a PVA/H₂SO₄ gel electrolyte was prepared as previously reported.⁷¹ 5 g H₂SO₄ was mixed with 50 mL DI water and then 5 g PVA powder was added to the acid solution. The whole mixture was heated to 80° C. with vigorous stirring until the solution became clear. Secondly, PANI was electrodeposited as-prepared deformable porous elastic conductor of Example 1 via electropolymerization of aniline at a potential of 0.8 V for 15 min in an aqueous solution of aniline (0.1 M) and H₂SO₄ (1 M) where KCl-saturated Ag/AgCl served as reference electrode and platinum wire as the counter electrode.

Then, the PANI/v-AuNWs deformable porous elastic conductor was immersed into PVA/H₂SO₄ gel electrolyte until the PANI/v-AuNWs deformable porous elastic conductor was saturated. Two pieces of PANI/v-AuNWs deformable porous elastic conductor were each placed onto separate sputtered Au PET substrate films, respectively. Then a piece filter paper saturated with electrolytes was placed onto one of the PANI/v-AuNWs sponge deformable porous elastic conductors. Both of the PANI/AuNWs deformable porous elastic conductors were then left in a fume hood for several hours. After that, the two deformable porous elastic conductors were pressed together and left at room temperature for another several hours, to produce an all-solid-state PANI/v-AuNWs soft supercapacitor comprising the deformable porous elastic conductor of the present invention. The volume of the supercapacitor was 1 cm×1 cm×0.3 cm.

### Example 4—Electrocatalysts; Recyclable Catalysis of 4-Nitrophenol to 4-Aminophenol by Porous Elastic Conductor

15 mL of freshly prepared 0.264 M sodium borohydride and 10 mL of 2.5 mM p-nitrophenol were mixed together in a beaker. The beaker was kept in a 45° C. oven for 10 min, then a piece of v-AuNWs deformable porous elastic conductor was immersed into the solution for 15 min and 0.5 mL of the solution was extracted for further UV-vis absorption analysis at certain intervals. The above process was repeated 10 times to investigate the recycled efficiency of the gold nanowire sponge as the catalyst.

### Example 5—Soft Battery-Free Wireless Pressure Sensing Antennae

Part a—v-AuNWs Sponge Ribbon

Polyurethane (PU) sponge was cut into spiral shape ribbons and washed with ethanol three times, followed by completely drying at 60° C. for 3 hours. Then the ribbon was placed into an air plasma chamber for 10 minutes treatment to render the ribbon surface hydrophilic. Subsequently, the ribbon was immersed into an ethanol solution of APTMS (5 mM) for two hours. When the immersion was complete, the ribbon was washed with ethanol and fully dried in oven (60° C., 3 hours).

Meanwhile, gold seed solutions were prepared in accordance with the procedure of example 1. The dried PU sponge ribbon was then immersed in the gold seed solution for another two hours to anchor gold seeds on the sponge's skeleton. After this step the ribbon was rinsed with DI water and dried in oven (65° C., 8 hours). Later, the ribbon was put into AuNWs growth solution of ethanol/water (v/v=1:1.2), including HAuCl₄ (12 mM), ligand MBA (1.1 mM), and reducing agent L-AA (30 mM) for 15 minutes. Then the sample was washed with ethanol and dried in oven (60° C., 3 hours).

### Part B—Elastomer Encapsulation

PDMS elastomer base and curing agent were mixed weight to weight with a ratio of 10:1, 20:1, 30:1 respectively and smeared on one side of the ribbon.

After curing at 65° C. for 15 minutes, the ribbon can be winded as a spiral as half-cured PDMS is tacky. After curing again for 4 hours at 65° C., the PDMS can bind the ribbon tightly and isolate it turn by turn. Then the sample was put into a petri dish (inner diameter is 52 mm, height is 12 mm) filled with uncured PDMS. The PDMS liquid can fully penetrate into sponge by degassing for 1 hour. Optionally, this step may be conducted in the presence of the application of sonication and/or the application of negative pressure to facilitate infiltration or penetration of the uncured elastomer into the 3D porous elastomeric substrate. Finally, the sample was put into oven and cured for 4 hours at 65° C.

For Ecoflex encapsulation, Part A and Part B of EcoFlex 00-30 were completely mixed with a weight ratio of 1:1. The encapsulation process of Ecoflex for sponge ribbon follows a procedure analogous to that used for PDMS encapsulation.

### Example 6—Fabrication of Deformation Insensitive Biophysiological Monitoring Electrode for ECG Monitoring

The deformable porous elastic conductor of example 1 was cut into 1 cm diameter cylindrical shaped electrodes or 1 cm×1 cm square shaped electrodes using a pair of scissors or a knife or a lever punch. Conductive adhesives such as silver paint, carbon black paint, and silver epoxy glue were applied on the contact pads of the flexible ECG device. The cylindrical shaped or square shaped porous elastic electrodes were allowed to dry for 2 hours before using.

The foregoing examples demonstrate that the deformable porous elastic conductors of the present invention exhibit exceptional conductivity (in terms of low resistance and/or low impedance and/or high current flow) that is surprisingly insensitive to harsh deformation environments, including deformation under tensile strain, compressive strain, twisting strain, or bending strain, whilst also being surprisingly insensitive to other potential sources of environmental damage or deterioration, including being insensitive to chemical damage in the form of aqueous solutions of surfactants or detergents, as well as being insensitive to physical damage of the kind likely to be encountered when used as a soft sensor in biomedical or biophysiological applications, such as physical damage due to scratching or rubbing or stripping of the surface of the deformable porous elastic conductor. Meanwhile, the deformable porous elastic conductors of the present invention exhibit a useful and tunable linear range of sensitivity, measured in terms of relative change in resistance with strain (ΔR/Rₒ) and/or relative change in current flow (ΔI/Iₒ), in response to tensile strain, and/or compressive strain, that advantageously makes them highly amenable to tensile strain sensing and pressure sensing applications,

### GENERAL

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

It should be appreciated that throughout this specification, any reference to any prior publication, including prior patent publications and non-patent publications, is not an acknowledgment or admission that any of the material contained within the prior publication referred to was part of the common general knowledge as at the priority date of the application.

Any manufacturer's instructions, descriptions, product specifications, and product sheets for any products mentioned herein or in any document incorporated by reference herein, are hereby incorporated herein by reference, and may be employed in the practice of the invention.

The invention described herein may include one or more range of values (eg. size, displacement and field strength etc). A range of values will be understood to include all values within the range, including the values defining the range, and values adjacent to the range which lead to the same or substantially the same outcome as the values immediately adjacent to that value which defines the boundary to the range.

The present invention is not to be limited in scope by any of the specific embodiments described herein. These embodiments are intended for the purpose of exemplification only. Functionally equivalent products, formulations and methods are clearly within the scope of the invention as described herein.

Those skilled in the art will appreciate that the invention described herein is susceptible to variations and modifications other than those specifically described. The invention includes all such variations and modifications. The invention also includes all of the steps, features, formulations and compounds referred to or indicated in the specification, individually or collectively and any and all combinations or any two or more of the steps or features.

### REFERENCES

1. B. Zhu, S. Gong and W. Cheng, Chem.Soc.Rev., 2019, 48, 1668-1711.
2. S. Gong, L. W. Yap, B. Zhu and W. Cheng, Adv. Mater., 2019, DOI: 10.1002/adma.201902278.
3. D. J. Lipomi, M. Vosgueritchian, B. C. Tee, S. L. Hellstrom, J. A. Lee, C. H. Fox and Z. Bao, Nat. Nanotechnol., 2011, 6, 788-792.
4. R. C. Webb, A. P. Bonifas, A. Behnaz, Y. Zhang, K. J. Yu, H. Cheng, M. Shi, Z. Bian, Z. Liu, Y. S. Kim, W. H. Yeo, J. S. Park, J. Song, Y. Li, Y. Huang, A. M. Gorbach and J. A. Rogers, Nat Mater, 2013, 12, 938-944.
5. A. Miyamoto, S. Lee, N. F. Cooray, S. Lee, M. Mori, N. Matsuhisa, H. Jin, L. Yoda, T. Yokota, A. Itoh, M. Sekino, H. Kawasaki, T. Ebihara, M. Amagai and T. Someya, Nat. Nanotechnol., 2017, 12, 907-913.
6. Y. Wang, S. Gong, S. J. Wang, X. Yang, Y. Ling, L. W. Yap, D. Dong, G. P. Simon and W. Cheng, ACS Nano, 2018, 12, 9742-9749.
7. S. Gong, L. W. Yap, B. Zhu, Q. Zhai, Y. Liu, Q. Lyu, K. Wang, M. Yang, Y. Ling, D. T. H. Lai, F. Marzbanrad and W. Cheng, Adv. Mater., 2019, DOI: 10.1002/adma.201903789.
8. Y. Tang, S. Gong, Y. Chen, L. W. Yap and W. Cheng, ACS Nano, 2014, 8, 5707-5714.
9. J. Ge, H. B. Yao, X. Wang, Y. D. Ye, J. L. Wang, Z. Y. Wu, J. W. Liu, F. J. Fan, H. L. Gao, C. L. Zhang and S. H. Yu, Angew. Chem., 2013, 52, 1968-1703.
10. Y. Yu, J. Zeng, C. Chen, Z. Xie, R. Guo, Z. Liu, X. Zhou, Y. Yang and Z. Zheng, Adv. Mater., 2014, 26, 810-815.
11. H. Chen, Z. Su, Y. Song, X. Cheng, X. Chen, B. Meng, Z. Song, D. Chen and H. Zhang, Adv. Funct. Mater., 2017, 27, 1604434.
12. Y. H. Wu, H. Z. Liu, S. Chen, X. C. Dong, P. P. Wang, S. Q. Liu, Y. Lin, Y. Wei and L. Liu, ACS Appl. Mater. Interfaces, 2017, 9, 20098-20105.
13. Z. Niu, W. Zhou, X. Chen, J. Chen and S. Xie, Adv. Mater., 2015, 27, 6002-6008.
14. W. Chen, R. B. Rakhi, L. Hu, X. Xie, Y. Cui and H. N. Alshareef, Nano Lett., 2011, 11, 5165-5172.
15. X. Wu, Y. Han, X. Zhang, Z. Zhou and C. Lu, Adv. Funct. Mater., 2016, 26, 6246-6256.
16. S. Chun, A. Hong, Y. Choi, C. Ha and W. Park, Nanoscale, 2016, 8, 9185-9192.
17. H. B. Yao, J. Ge, C. F. Wang, X. Wang, W. Hu, Z. J. Zheng, Y. Ni and S. H. Yu, Adv. Mater., 2013, 25, 6692-6698.
18. H. Zhang, N. Liu, Y. Shi, W. Liu, Y. Yue, S. Wang, Y. Ma, L. Wen, L. Li, F. Long, Z. Zou and Y. Gao, ACS Appl. Mater. Interfaces, 2016, 8, 22374-22381.
19. Y. Tang, K. L. Yeo, Y. Chen, L. W. Yap, W. Xiong and W. Cheng, J. Mater. Chem. A, 2013, 1, 6723-6726.
20. J. Y. Oh, D. Lee and S. H. Hong, ACS Appl. Mater. Interfaces, 2018, 10, 21666-21671.
21. L. Xie, F. Su, L. Xie, X. Li, Z. Liu, Q. Kong, X. Guo, Y. Zhang, L. Wan, K. Li, C. Lv and C. Chen, ChemSusChem, 2015, 8, 2917-2926.
22. Y. Wang, D. Kong, W. Shi, B. Liu, G. J. Sim, Q. Ge and H. Y. Yang, Advanced Energy Materials, 2016, 6, 1601057.
23. J. Kim, M. Kim, M. S. Lee, K. Kim, S. Ji, Y. T. Kim, J. Park, K. Na, K. H. Bae, H. K. Kim, F. Bien, C. Y. Lee and J. U. Park, Nat. Commun., 2017, 8, 14997.
24. L. Y. Chen, B. C. K. Tee, A. L. Chortos, G. Schwartz, V. Tse, D. J. Lipomi, H. S. P. Wong, M. V. McConnell and Z. Bao, Nat. Commun., 2014, 5, 5028.
25. D. Lu, Y. Yan, Y. Deng, Q. Yang, J. Zhao, M. H. Seo, W. Bai, M. R. MacEwan, Y. Huang, W. Z. Ray and J. A. Rogers, Adv. Funct. Mater., 2020, 30, 2003754.
26. H. Chang, S. Kim, T. H. Kang, S. W. Lee, G. T. Yang, K. Y. Lee and H. Yi, ACS Appl. Mater. Interfaces, 2019, 11, 32291-32300.
27. K. Meng, Y. Wu, Q. He, Z. Zhou, X. Wang, G. Zhang, W. Fan, J. Liu and J. Yang, ACS Appl. Mater. Interfaces, 2019, 11, 46399-46407.
28. B. Nie, R. Huang, T. Yao, Y. Zhang, Y. Miao, C. Liu, J. Liu and X. Chen, Adv. Funct. Mater., 2019, 29, 1808786.
29. G. H. Lee, J. K. Park, J. Byun, J. C. Yang, S. Y. Kwon, C. Kim, C. Jang, J. Y. Sim, J. G. Yook and S. Park, Adv. Mater., 2020, 32, 1906269.
30. F. Melloni, G. E. Bonacchini, G. Lanzani and M. Caironi, Adv. Mater. Technol., 2020, 5, 2000389.
31. R. Sun, S. C. Carreira, Y. Chen, C. Xiang, L. Xu, B. Zhang, M. Chen, I. Farrow, F. Scarpa and J. Rossiter, Adv. Mater. Technol., 2019, 4, 1900100.
32. A. Palmroth, T. Salpavaara, J. Lekkala and M. Kellomaki, Adv. Mater. Technol., 2019, 4, 1900428.
33. W. Hyung, B. Oh, S. Kim, J. Jang, S. Ji, S. Lee, J. Cheon, S. Yoo, S. Lee and J. Park, Nano Energy, 2019, 62, 230-238.
34. Q. Wang, L. Jiang, Y. Yu and J. Sun, Nano Energy, 2019, 55, 93-114.
35. Q. A. Huang, L. Dong and L. F. Wang, J. Microelectromechanical Syst., 2016, 25, 822-841.
36. R. Wu, L. Ma, A. Patil, C. Hou, S. Zhu, X. Fan, H. Lin, W. Yu, W. Guo and X. Y. Liu, ACS Appl. Mater. Interfaces, 2019, 11, 33336-33346.
37. A. Palmroth, T. Salpavaara, P. Vuoristo, S. Karjalainen, T. Kssrisinen, S. Miettinen, J. Massera, J. Lekkala and M. Kellomaki, ACS Appl. Mater. Interfaces, 2020, 12, 31148-31161.
38. C. M. Boutry, L. Beker, Y. Kaizawa, C. Vassos, H. Tran, A. C. Hinckley, R. Pfattner, S. Niu, J. Li, J. Claverie, Z. Wang, J. Chang, P. M. Fox and Z. Bao, Nat. Biomed. Eng., 2019, 3, 47-57.
39. P. Chen, S. Member, S. Member, D. C. Rodger, S. Saati, M. S. Humayun and Y. Tai, J. Microelectromechanical Syst., 2008, 17, 1342-1351.
40. J. Park, J. K. Kim, D. S. Kim, A. Shanmugasundaram, S. A. Park, S. Kang, S. H. Kim, M. H. Jeong and D. W. Lee, Sensors Actuators, B Chem., 2019, 280, 201-209.
41. Q. Tan, W. Lv, Y. Ji, R. Song, F. Lu, H. Dong, W. Zhang and J. Xiong, Sensors Actuators, B Chem., 2018, 270, 433-442.
42. R. Rahimi, M. Ochoa and B. Ziaie, ACS Appl. Mater. Interfaces, 2016, 8, 16907-16913.
43. A. R. Mohammadi, T. C. M. Graham, C. P. J. Bennington and M. Chiao, Sensors Actuators, A Phys., 2010, 163, 471-480.
44. J. Xiong, Y. Li, Y. Hong, B. Zhang, T. Cui, Q. Tan, S. Zheng and T. Liang, Sensors Actuators, A Phys., 2013, 197, 30-37.
45. Cardiovascular Disease 2016, accessed 18 Sep. 2019, Australian Government Department of Health, http://www.health.gov.au/internet/main/publishing.nsf/content/chronic-cardio
46. Atrial Fibrillation, accessed 18 Sep. 2019, The Heart Foundation, https://www.heartfoundation.org.au/your-heart/heart-conditions/atrial-fibrillation
47. Heart conditions—atrial fibrillation, accessed 18 Sep. 2019, Better Health Channel, https://www.betterhealth.vic.gov.au/health/conditionsandtreatments/heart-conditions-atrial-fibrillation
48. M. A. Lopez-Gordo, D. Sanchez-Morillo and F. Pelayo Valle. Dry EEG Electrodes, Sensors 2014, 14(7), 12847-12870.
49. Shing-Hong Liu. Motion Artifact Reduction in Electrocardiogram Using Adaptive Filter, J. Med. Biol. Eng. 2011, 31(1), 67-72
50. Shuto Nagai, Daisuke Anzai and Jianqing Wang. Motion artefact removals for wearable ECG using stationary wavelet transform, Healthc Technol Lett. 2017, 4(4), 138-141
51. Z. Niu, H. Dong, B. Zhu, J. Li, H. H. Hng, W. Zhou, X. Chen and S. Xie, Adv. Mater., 2013, 25, 1058-1064.
52. W. Liu, Z. Chen, G. Zhou, Y. Sun, H. R. Lee, C. Liu, H. Yao, Z. Bao and Y. Cui, Adv. Mater., 2016, 28, 3578-3583.
53. S. Liang, Y. Li, J. Yang, J. Zhang, C. He, Y. Liu and X. Zhou, Adv. Mater. Technol., 2016, 1, 1600117.
54. M. Chen, S. Duan, L. Zhang, Z. Wang and C. Li, Chem. Commun., 2015, 51, 3169-3172.
55. I. K. Moon, S. Yoon and J. Oh, Adv. Mater. Interfaces, 2017, 4, 1700860.
56. X. Huang, Y. Liu, H. Cheng, W. Shin, J. A. Fan, Z. Liu, C. Lu, G. Kong, K. Chen, D. Patnaik, S. Lee, S. Hage-ali, Y. Huang and J. A. Rogers, Adv. Funct. Mater., 2014, 24, 3846-3854.
57. E. Ban, H. Wang, J. Matthew Franklin, J. T. Liphardt, P. A. Janmey and V. B. Shenoy, Proc. Natl. Acad. Sci. U.S.A., 2019, 116, 6790-6799.
58. Q. Tan, T. Luo, J. Xiong, H. Kang, X. Ji, Y. Zhang, M. Yang, X. Wang, C. Xue, J. Liu and W. Zhang, Sensors (Switzerland), 2014, 14, 4154-4166.
59. Y. Ling, T. An, L. W. Yap, B. Zhu, S. Gong and W. Cheng, Adv. Mater., 2020, 32, 1904664.
60. S. Gong and W. Cheng, Adv. Energy Mater., 2017, 7, 1700648.
61. C. I. Jang, K. S. Shin, M. J. Kim, K. S. Yun, K. H. Park, J. Y. Kang and S. H. Lee, Appl. Phys. Lett., DOI:10.1063/1.4943136.
62. Y. W. Kim, M. J. Kim, K. H. Park, J. W. Jeoung, S. Hwan, C. In, J. Ms, S. H. Lee, J. H. Kim, S. Lee and J. Yoon, Clin. Exp. Ophthalmol., 2015, 830-837.

1. A deformable porous elastic conductor comprising;
a 3D porous elastomeric substrate, wherein a plurality of the surfaces of the 3D porous elastomeric substrate are covalently functionalised with complexing moieties; and
a plurality of metal nanowires, each complexed to at least one of the complexing moieties, wherein the metal nanowires are upstanding, relative to the surface to which they are attached via their respective complexing moiety.

2. The deformable porous elastic conductor according to claim 1, wherein the metal nanowires comprise a nanoparticle head and a nanowire tail.

3. The deformable porous elastic conductor according to claim 1, wherein the metal nanowires comprise;
(i) a metal selected from the group consisting of gold, platinum, palladium, rhodium, copper, silver, ruthenium, osmium, iridium, rhenium, iron, cobalt, nickel, zinc, manganese, titanium, vanadium, chromium, molybdenum, tungsten, magnesium, lead and aluminium; and/or
(ii) a noble metal; and/or
(iii) gold.

4. The deformable porous elastic conductor according to claim 1 which is;
(i) compressible; and/or
(ii) biocompatible; and/or
(iii) chemically inert.

5. The deformable porous elastic conductor according to claim 1, wherein;
(i) the 3D porous elastomeric substrate is a sponge, or a synthetic polymer sponge, or a polyurethane sponge; and/or
(ii) the complexing moieties are amine groups.

6. The deformable porous elastic conductor according to claim 1, wherein;
(i) the plurality of the surfaces of the 3D porous elastomeric substrate are covalently functionalised with an (Aminoalkyl)trialkyloxysilane, or (3-Aminopropyl)trimethoxysilane or (3-Aminopropyl)triethoxysilane; and/or
(ii) the plurality of the surfaces of the 3D porous elastomeric substrate are covalently functionalised with an alcoholic solution of (Aminoalkyl)trialkyloxysilane, or (3-Aminopropyl)trimethoxysilane or (3-Aminopropyl)triethoxysilane; and/or
(iii) the plurality of the surfaces of the 3D porous elastomeric substrate are covalently functionalised with an aqueous solution of (Aminoalkyl)trialkyloxysilane, or (3-Aminopropyl)trimethoxysilane or (3-Aminopropyl)triethoxysilane.

7. The deformable porous elastic conductor according to claim 1;
(i) having a conductivity which is insensitive to tension, compression, bending or twisting; or
(ii) having a linear region of response to strain when measured as relative change in resistance (ΔR/Rₒ) with strain or relative change in current (ΔI/Iₒ) with strain.

8. The deformable porous elastic conductor according to claim 1 having either;
A:
(i) a conductivity of 1500 S m⁻¹ or better, preferably a conductivity of 5500 S m⁻¹ or better; and/or
(ii) insensitivity to tensile strain as measured by relative resistance (R/Rₒ) of 15% or less at up to 44% strain; and/or
(iii) insensitivity to compressive strain as measured by relative change in resistance (ΔR/Rₒ), of 42% or less at up to 80% compressive strain; and/or
(iv) insensitivity to bending as measured by relative change in resistance (ΔR/Rₒ), of 8% or less at up to 180° bending; and/or
(v) insensitivity to twisting as measured by relative change in resistance (ΔR/Rₒ), of 21% or less at up to 1080° twisting; and/or
(vi) insensitivity to washing with aqueous detergent solution as measured by relative change in resistance (ΔR/Rₒ), of 26% or less at up to 10 cycles of washing with aqueous detergent solution; and/or
(vii) insensitivity to tape stripping tests as measured by relative change in resistance (ΔR/Rₒ), of 14% or less at up to 10 cycles of tape stripping test; and/or
(viii) insensitivity to scratch tests as measured by relative change in resistance (ΔR/Rₒ), of 41% or less at up to 10 cycles of scratch test; and/or
(ix) insensitivity to rubbing tests as measured by relative change in resistance (ΔR/Rₒ), of 50% or less at up to 10 cycles of rubbing test; or
B:
(i) a linear region of response to tensile strain when measured as relative change in resistance with strain (ΔR/Rₒ), in the range of 30-50% tensile strain, or 50-70% tensile strain, or 10-70% tensile strain; and/or
(ii) a linear region of response to compressive strain when measured as relative change in current (ΔI/Iₒ) with compressive strain, in the range of 5 kPa to 38 kPa; preferably with a sensitivity within the linear region of 8.42 kPa⁻¹.

9. The deformable porous elastic conductor according to claim 1, embedded in a solid elastomeric material, PDMS elastomer, or an addition cure silicone rubber, preferably wherein the embedded deformable porous elastic conductor is;
(i) insensitive to tensile strain as measured by relative resistance (R/R₀) of 1.3 or less at up to 60% strain and 1.9 or less at up to 100% strain; and/or
(ii) stretchable up to approximately 340% without loss of conductivity and/or without significant deterioration in conductivity; and/or
(iii) highly durable, as determined by 12% or less changes in conductivities under 5000 stretch-release cycles at 30% strain.

10. The deformable porous elastic conductor according to claim 1 when used as a soft electronic device, or a sensor, or a wearable sensor, or a soft inductive-capacitive sensor, or a dry soft electrode, or a biophysiological monitoring electrode.

11. An electrode, a biophysiological monitoring electrode, a supercapacitor, an antenna, or an electrocatalyst comprising the deformable porous elastic conductor according to claim 1.

12. A device, selected from the group comprising a data collection device, a biophysiological monitoring device, an Electrocardiograph (ECG) device, an Electromyograph (EMG) device, and an Electroencephalograph (EEG) device, comprising the deformable porous elastic conductor according to claim 1; optionally wherein the device is wearable, and capable of wirelessly transmitting data to a separate data logging and processing device.

13. The device of claim 12, wherein the deformable porous elastic conductor or the electrode;
(i) maintains a stable electrical resistance of 1Ω for over 30 days of use; and/or
(ii) has a thickness of approximately 2 mm, or a thickness of less than approximately 2 mm, or a thickness of approximately 1.5 mm, or a thickness of less than approximately 1.5 mm, or a thickness of approximately 1 mm, or a thickness of less than approximately 1 mm.

14. The device of claim 12, wherein the device;
(i) comprises an ultrathin battery, having a thickness of not more than 1 mm; and/or
(ii) comprises a flexible circuit board, comprising at least one microprocessor and a wireless transmitter; and/or
(iii) comprises a soft flexible adhesive for attaching the device to a user, or a subject, or a surface from which data is to be collected; and/or
(iv) is not more than 6.1 cm long, not more than 2.6 cm wide and not more than 4 mm thick; and/or
(v) is reusable, cleanable and sanitisable.

15. A method of fabricating the deformable porous elastic conductor of claim 1, the method comprising the steps of;
(i) optionally, pre-treating the 3D porous elastomeric substrate; preferably via air plasma treatment;
(ii) functionalising the 3D porous elastomeric substrate with a functionalising agent; preferably via introducing a functionalising agent in the presence of the application of sonication and/or the application of negative pressure to facilitate infiltration or penetration of the functionalising agent into the 3D porous elastomeric substrate; preferably wherein the functionalising agent is;
a) an (Aminoalkyl)trialkyloxysilane, or (3-Aminopropyl)trimethoxysilane, or (3-Aminopropyl)triethoxysilane; and/or
b) an alcoholic solution of an (Aminoalkyl)trialkyloxysilane, or (3-Aminopropyl)trimethoxysilane, or (3-Aminopropyl)triethoxysilane; and/or
c) an aqueous solution of an (Aminoalkyl)trialkyloxysilane, or (3-Aminopropyl)trimethoxysilane, or (3-Aminopropyl)triethoxysilane;
(iii) seeding the functionalised 3D porous elastomeric substrate with metal nanoparticles; preferably via introducing a seed solution comprising metal nanoparticles and optionally a stabiliser, optionally in the presence of the application of sonication and/or the application of negative pressure to facilitate infiltration or penetration of the seed solution into the 3D porous elastomeric substrate; preferably wherein the metal nanoparticles are noble metal nanoparticles; most preferably wherein the metal nanoparticles are gold nanoparticles; and
(iv) growing metal nanowires from the metal nanoparticles; preferably via introducing a growth solution comprising a metal salt, a reducing agent and a surfactant or ligand, optionally in the presence of the application of sonication and/or the application of negative pressure to facilitate infiltration or penetration of the growth solution into the 3D porous elastomeric substrate; preferably wherein the metal nanowires are gold nanowires and the metal salt is HAuCl₄; and/or preferably wherein the reducing agent is L-ascorbic acid; and/or preferably wherein the surfactant or ligand is 4-mercaptobenzoic acid; optionally wherein the growth of the nanowires is tuned by fabricating a series of the deformable porous elastic conductors with varying concentrations of growth solution.
